(19) United States
(12) Patent Application Publication
(10) Pub. No.: US 2026/0191402 A1
(43) Pub. Date: Jul. 9, 2026

(54) COVERINGS FOR BENDABLE ENDOSCOPE SYSTEMS

(71) Applicant: COOK MEDICAL TECHNOLOGIES LLC, Bloomington, IN (US)

(72) Inventors: Liam Breen, Ballina (IE); Ahmed Wanas, Limerick (IE); Ciara Kavanagh, Surry Hill (AU); Evan Creaven, Ballina (IE); Katherine Shaw, Limerick (IE)

(21) Appl. No.: 19/442,246

(22) Filed: Jan. 7, 2026

Related U.S. Application Data
(60) Provisional application No. 63/743,114, filed on Jan. 8, 2025.

(51) Int. Cl. A61B 1/005; A61B 1/00; A61B 1/018

(52) U.S. Cl. CPC A61B 1/0055; A61B 1/00119; A61B 1/00137; A61B 1/018

(57) ABSTRACT

Endoscope systems with bending sections that can bend in four directions independent of the rotatable viewing configuration of the endoscope systems are provided. Methods of using the endoscope systems in a patient’s body are further provided. The distal ends of the endoscope systems may have one or more sleeves, coverings, and/or patches surrounding at least a portion thereof.

### PRIORITY CLAIM

The present patent document claims the benefit of priority of U.S. Provisional Application Serial No. 63/743,114, filed January 8, 2025, which is hereby fully incorporated by reference in its entirety.

### FIELD

The present disclosure relates to medical devices. More particularly, the present disclosure relates to distal end coverings for bendable endoscope systems.

### BACKGROUND

The statements in this section merely provide background information related to the present disclosure and may not constitute prior art.

The duodenoscope is a medical device used in a variety of endoscopic procedures, including endoscopic retrograde cholangiopancreatography (“ERCP”). In an ERCP, a physician inserts the duodenoscope into a patient’s mouth, through the patient’s gastrointestinal (“GI”) tract, and into the duodenum until the distal end of the duodenoscope is positioned near the papilla of Vater, a small mound-like structure that acts as the entrance from the common bile duct and pancreatic duct into the duodenum. The physician then uses a variety of tools and accessories that are passed through a lumen in the duodenoscope to access the common bile duct or pancreatic duct through the papilla of Vater.

However, the duodenoscope suffers from several design issues. For example, due to the location of the papilla of Vater and shape of the duodenoscope, the endoscope tools or accessories must be bent sharply at (or sometimes more than) 90 degree angles at the distal end of the duodenoscope, which results in significant friction between the tools and duodenoscope and accompanying force transmission loss. Therefore, the accessories must be durable enough to withstand this sharp bend and the physician must apply a greater force to continue to advance the tools than is desired. Further, the built-in camera system of the duodenoscope is side-facing, making it difficult for novices and even experienced physicians to navigate the duodenoscope through the GI tract. Also, traditional duodenoscopes only have one accessory channel, making the use of multiple accessories time intensive and cumbersome. Additionally, duodenoscopes are difficult to clean, which may result in inadequate cleaning of the device after use and potential bacterial contamination of patients during subsequent use of the duodenoscope.

It is desirable to have an endoscope system that eliminates or lessens the force transmission losses of duodenoscopes. Increased and easier maneuverability of an endoscope system through and within the GI tract is desired.

All endoscopes should include a mechanism to bend the distal end so that the physician can steer the endoscope to scan the GI lumen and navigate the GI anatomy. Links of the bending section are intact around the entire circumference of the distal-proximal longitudinal axis of the bending section. However, such bending sections do not allow the accessory channels to bend outwards so that the bending section can advantageously switch between a forward-viewing perspective and a side-viewing perspective. Further, such bending sections may be experimentally inefficient in deflecting with proper degrees of freedom to cannulate a duodenum. Accordingly, for certain practical applications, 4-way deflection of links with “U”-shaped profiles may be preferable. Thus, there remains a need for further contributions in this area of technology.

### SUMMARY

In one form of the present disclosure, a scope system is provided.

In one aspect, a scope system has an elongate tube comprising a lumen extending therethrough, the elongate tube further comprising a distal portion having a plurality of circumferentially uncontinuous ribs, each of the circumferentially uncontinuous ribs comprising a rib opening. At least one accessory channel comprises a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration. A sleeve circumferentially surrounds at least the plurality of circumferentially discontinuous ribs, the sleeve having a longitudinal slot corresponding to the rib openings of the plurality of circumferentially discontinuous ribs. During rotation of the distal end section relative to the distal portion, the at least one accessory channel is reversibly removable from an interior cavity of the plurality of circumferentially uncontinuous ribs through the rib openings and the longitudinal slot.

In another aspect, a scope system has an elongate tube comprising a lumen extending therethrough, the elongate tube further comprising a distal portion having a plurality of circumferentially uncontinuous ribs, each of the circumferentially uncontinuous ribs comprising a rib opening. At least one accessory channel comprises a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration. An inner cover surrounds at least a portion of a periphery of the at least one accessory channel positioned within the plurality of circumferentially uncontinuous ribs. During rotation of the distal end section relative to the distal portion, the at least one accessory channel and the inner covering are reversibly removable from an interior cavity of the plurality of circumferentially uncontinuous ribs through the rib openings.

In a further aspect, a scope system has an elongate tube comprising a lumen extending therethrough, the elongate tube further comprising a distal portion having a plurality of circumferentially uncontinuous ribs, each of the circumferentially uncontinuous ribs comprising a rib opening. At least one accessory channel comprises a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration. An elastic sleeve circumferentially surrounds at least the plurality of circumferentially discontinuous ribs. During rotation of the distal end section relative to the distal portion, the elastic sleeve deforms as the at least one accessory channel is reversibly removed from an interior cavity of the plurality of circumferentially uncontinuous ribs through the rib openings.

In yet another aspect, a scope system includes an elongate tube comprising a lumen extending therethrough, the elongate tube further comprising a distal portion having a plurality of circumferentially uncontinuous ribs, each of the circumferentially uncontinuous ribs comprising a rib opening. At least one accessory channel comprises a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration. The scope system further includes at least one or more of:

a) a sleeve circumferentially surrounding at least the plurality of circumferentially discontinuous ribs, the sleeve having a longitudinal slot corresponding to the rib openings of the plurality of circumferentially discontinuous ribs, wherein, during rotation of the distal end section relative to the distal portion, the at least one accessory channel is reversibly removable from an interior cavity of the plurality of circumferentially uncontinuous ribs through the rib openings and the longitudinal slot;

b) an inner cover surrounding at least a portion of a periphery of the at least one accessory channel positioned within the plurality of circumferentially uncontinuous ribs, wherein, during rotation of the distal end section relative to the distal portion, the at least one accessory channel and the inner covering are reversibly removable from the interior cavity of the plurality of circumferentially uncontinuous ribs through the rib openings; and/or,

c) an elastic sleeve circumferentially surrounding at least the plurality of circumferentially discontinuous ribs, wherein, during rotation of the distal end section relative to the distal portion, the elastic sleeve deforms as the at least one accessory channel is reversibly removed from the interior cavity of the plurality of circumferentially uncontinuous ribs through the rib openings.

In another aspect, a scope system includes an elongate tube having a lumen extending therethrough, the elongate tube further comprising a distal portion having a plurality of circumferentially uncontinuous ribs, each of the circumferentially uncontinuous ribs comprising a rib opening. At least one accessory channel comprises a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration. A sleeve circumferentially surrounds at least the plurality of circumferentially discontinuous ribs, the sleeve having a longitudinal zone of weakness corresponding to the rib openings of the plurality of circumferentially uncontinuous ribs. During rotation of the distal end section relative to the distal portion, the at least one accessory channel is reversibly removable from an interior cavity of the plurality of circumferentially uncontinuous ribs through the rib openings and the longitudinal zone of weakness.

Further areas of applicability will become apparent from the description provided herein. It should be understood that the description and specific examples are intended for purposes of illustration only and are not intended to limit the scope of the present disclosure.

### DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts throughout the different views.

FIG. 1 illustrates a side view of an example of an endoscope system having a bending section according to the principles of the present disclosure;

FIG. 2 illustrates a detailed view of a distal portion of an example of an endoscope system in a forward-facing configuration;

FIGS. 3-5 illustrate views of an example of a circumferentially continuous, ring-shaped rib for an example of an endoscope system constructed according to the principles of the present disclosure;

FIGS. 6-8 illustrate views of an example of a circumferentially uncontinuous, C-shaped, or open rib for an example of an endoscope system constructed according to the principles of the present disclosure;

FIGS. 9A and 9B illustrate a view of the symmetry associated with an example of a circumferentially uncontinuous, C-shaped, or open rib for an example of an endoscope system constructed according to the principles of the present disclosure;

FIGS. 10 and 11 illustrate detailed views of an example of a distal portion of an endoscope system constructed in accordance with the principles of the present disclosure;

FIG. 12 illustrates another example of ribs of an example of an endoscope system of the present disclosure disposed along a series of control wires;

FIG. 13 illustrates a detailed view of an example of a pivot arm of an example of an endoscope system in a forward-facing configuration;

FIG. 14 illustrates a detailed view of an example of a pivot arm of an example of an endoscope system in a side-facing configuration;

FIG. 15 illustrates a detailed view of a distal portion of an example of an endoscope system in a side-facing configuration;

FIG. 16 illustrates a detailed view of a distal portion of an example of an endoscope system in a bent configuration;

FIG. 17 illustrates a detailed view of an example of an axially rotatable bearing of an example of an endoscope system;

FIG. 18 illustrates a detailed view of a first example of a distal portion of an endoscope system constructed in accordance with the principles of the present disclosure, having a sleeve surrounding at least a portion thereof;

FIG. 19 illustrates a top view of the embodiment of FIG. 18;

FIG. 20 illustrates the embodiment of FIG. 18, in a side-facing configuration;

FIG. 21 illustrates the embodiment of FIG. 18, in a side-facing configuration, shown in a partial phantom view;

FIG. 22 illustrates a detailed view of a second example of a distal portion of an endoscope system constructed in accordance with the principles of the present disclosure, having a sleeve and a patch surrounding at least a portion thereof;

FIG. 23 illustrates the embodiment of FIG. 22, in a side-facing configuration;

FIG. 24 illustrates the embodiment of FIG. 22, in a side-facing configuration, shown in a partial phantom view;

FIG. 25 illustrates a top view of the embodiment of FIG. 22;

FIGS. 25A-B illustrate cross-sectional views of FIG. 25;

FIG. 26 illustrates a detailed view of a third example of a distal portion of an endoscope system constructed in accordance with the principles of the present disclosure, having a sleeve and a patch surrounding at least a portion thereof;

FIG. 27 illustrates the embodiment of FIG. 26, in a side-facing configuration;

FIG. 28 illustrates the embodiment of FIG. 26, in a side-facing configuration, shown in a partial phantom view;

FIG. 29 illustrates a top view of the embodiment of FIG. 26;

FIGS. 29A-B illustrate cross-sectional views of FIG. 29;

FIG. 30 illustrates a detailed view of a fourth example of a distal portion of an endoscope system constructed in accordance with the principles of the present disclosure, having an inner cover surrounding at least a portion of at least one accessory channel;

FIG. 30A illustrates a cross-sectional view of FIG. 30;

FIG. 31 illustrates a detailed view of a fifth example of a distal portion of an endoscope system constructed in accordance with the principles of the present disclosure, having an inner cover surrounding at least a portion of at least one accessory channel;

FIG. 31A illustrates a cross-sectional view of FIG. 31;

FIG. 32 illustrates a detailed view of a sixth example of a distal portion of an endoscope system constructed in accordance with the principles of the present disclosure, having an elastic sleeve surrounding at least a portion thereof, and optionally, one or more of the sleeves, coverings, and patches illustrated in FIGS. 18 to 31;

FIG. 33 illustrates a seventh example of a distal portion of an endoscope system constructed in accordance with the principles of the present disclosure, having a sleeve surrounding at least a portion thereof;

FIG. 34 illustrates the embodiment of FIG. 33, in a side-facing configuration; and,

FIG. 35 illustrates an eighth example of a distal portion of an endoscope system constructed in accordance with the principles of the present disclosure, having a sleeve surrounding at least a portion thereof.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features. It should also be understood that various cross-hatching patterns used in the drawings are not intended to limit the specific materials that may be employed in the present disclosure. The cross-hatching patterns are merely exemplary of preferable materials or are used to distinguish between adjacent or mating components illustrated within the drawings for purposes of clarity.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer an axis that aligns with the proximal-distal axis 71 of the device (or component), for example, when the device is not bent. The terms “radially” and “radial” will be used to refer to elements, surfaces, or assemblies relative to one another that may extend perpendicularly from a longitudinal axis. The terms “circumference,” “circumferentially,” and “circumferential” will be used to elements, surfaces, or assemblies relative to one another encircling or substantially encircling a longitudinal axis at a radius.

The uses of the terms “a” and “an” and “the” and similar references in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of” the elements presented herein, whether explicitly set forth or not.

In describing elements of the present disclosure, the terms 1^{st}, 2^{nd}, first, second, A, B, (a), (b), and the like, may be used herein. These terms are only used to distinguish one element from another element, but do not limit the corresponding elements, irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

In the context of the present disclosure, a first piece is said to be integral to a second piece if the first and second pieces are formed as a single piece. For example, if the first and second pieces are cast as a single plastic piece, then the first piece is integral to the second piece.

As used herein, the term “about,” when used in the context of a numerical value or range set forth, means a variation of ±15%, or less, of the numerical value. For example, a value differing by ±15%, ±14%, ±10%, or ±5%, among others, would satisfy the definition of “about,” unless more narrowly defined in particular instances.

### BENDABLE ENDOSCOPE SYSTEMS

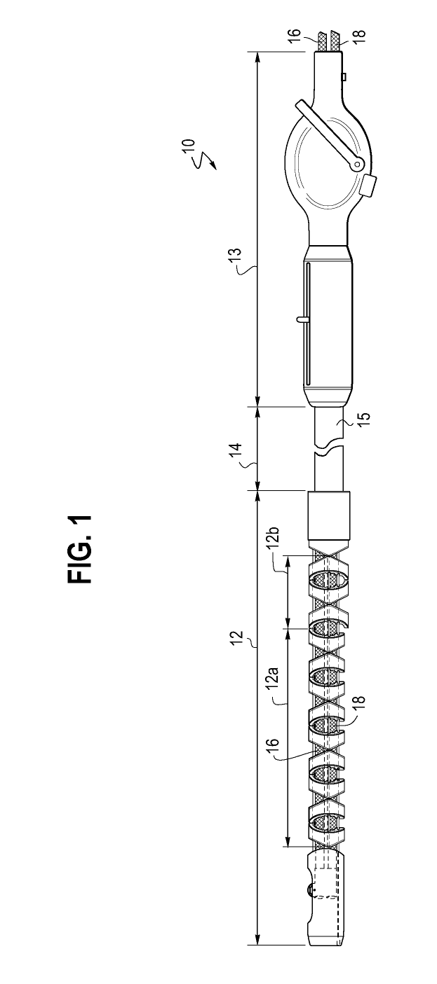

Referring to FIG. 1, an example of an endoscope system 10 is illustrated. The endoscope system 10 may be generally shaped as an elongate tube including a distal portion 12, a central portion 14, and a proximal, or handle, portion 13. The central portion 14 may be a flexible, elongate tube with at least one lumen 15 running throughout the length of the central portion 14. The central portion 14 may connect the distal portion 12 and proximal portion 13 together. The at least one lumen 15 of the central portion 14 may extend through the distal portion 12 and handle portion 13 of the endoscope system 10 as well. The central portion 14 may be made of a braided material, such as a polyether block amide (including, for example, PEBAX) with a polytetrafluoroethylene (“PTFE”) liner to provide sufficient torqueability and pushability. Other potential materials for the central portion 14 include, but are not limited to, polyethylene, polypropylene, and nylon.

The endoscope system 10 may further include two accessory channels 16, 18 each with a lumen running therethrough. The accessory channels 16, 18 may be designed as individual elongated tubes that may be movable within the at least one lumen 15 of the endoscope system 10, thus allowing longitudinal movement of the accessory channels 16, 18 with respect to the central portion 14. While this example includes two accessory channels 16, 18, one or even three or more accessory channels may be included. For example, a single, larger accessory channel may be used to accommodate larger endoscopic tools. Further, in lieu of individual accessory channels 16, 18, a single elongate tube may be used with two or more lumens running through it. The accessory channels 16, 18 may range in diameter anywhere from 1 to 10 millimeters. In one example, accessory channel 16 may be 4.2 millimeters in diameter while accessory channel 18 may be 3.7 millimeters in diameter. The accessory channels 16, 18 may extend proximally from or past the handle portion 13, through the at least one lumen 15 and into the distal portion 12. Various tools, devices, and cameras may be at least partially inserted into and removably disposed in the accessory channels 16, 18.

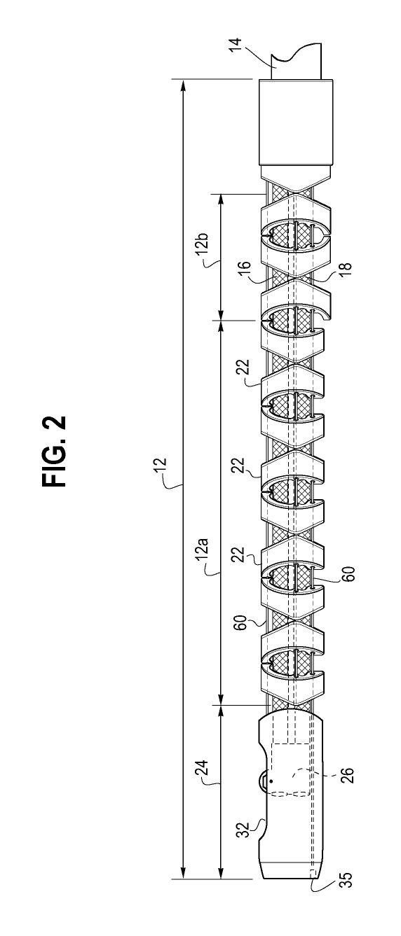

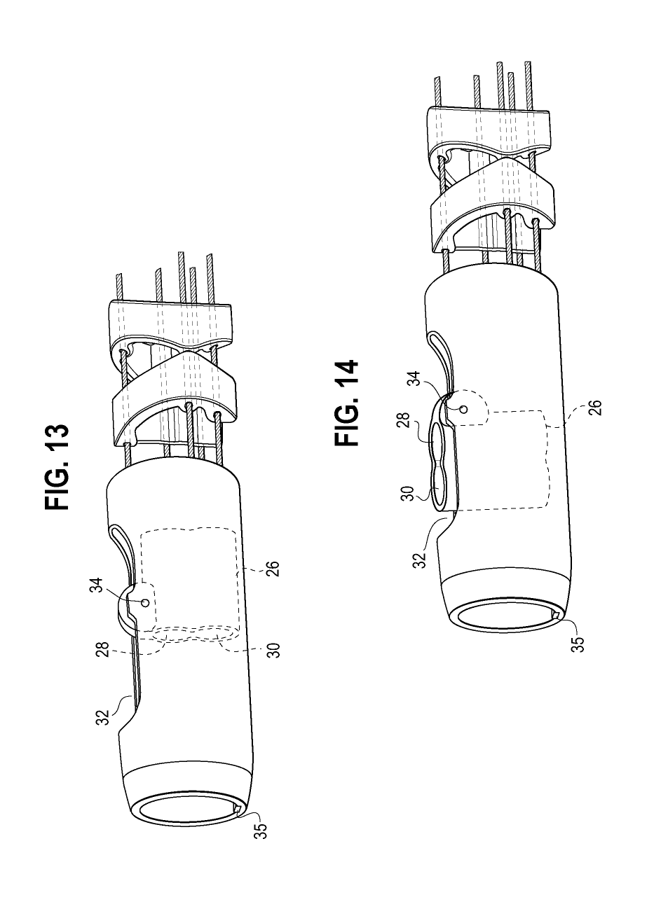

Now referring to FIG. 2, a detailed view of the distal portion 12 of an example of endoscope system 10 is illustrated. The endoscope system 10 may include an axially rotatable bearing 80 disposed between the central portion 14 and the distal portion 12, which may allow or permit the distal portion 12 to rotate independently of the central portion 14. The distal portion 12 may have a flexible rib-like construction with multiple individual ribs 22 connected together to create an elongate tube with at least one lumen 15. Ribs 22 may be made of a variety of materials, such as polycarbonate, nylon, polyethylene, polypropylene, and polyoxymethylene. The accessory channels 16, 18 may travel through the ribs 22 to the distal end section 24 of the distal portion 12. The distal end section 24 may include a pivot arm 26 with first and second accessory lumens 28, 30 (shown in FIGS. 13 and 14). The distal ends of the accessory channels 16, 18 may be fixedly or movably disposed within respective first and second accessory lumens 28, 30. The distal end section 24 may also include a side port 32 that provides access from the at least one lumen 15 to a point external the endoscope system 10.

An example of a distal end section 24 of a distal portion 12 is illustrated in more detail in FIGS. 13 and 14. For clarity, the accessory channels 16, 18 are omitted from FIGS. 13 and 14. The pivot arm 26 may be connected or coupled to the distal end section 24 via a pin 34. The pin 34 may create a pivot point, around which the pivot arm 26 may rotate or rotatably pivot with respect to the distal end section 24 to the position shown in FIG. 14. The pivot arm 26 may be moved between a forward-viewing position as shown in FIG. 13 and a side-viewing position as shown in FIG. 14. A light emitting diode (“LED”) light 35 may be placed on the distal end section 24 to assist in navigation through a patient’s GI tract. Alternatively, the LED light 35 may be placed at other locations on the distal end section 24, such as near the side port 32. Also, multiple LED lights 35 may be used at various locations on the endoscope system 10.

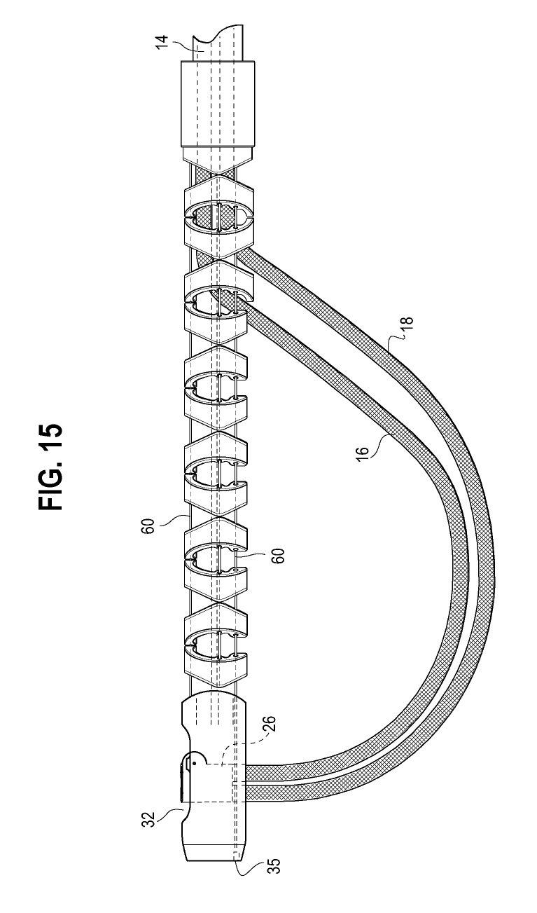

As illustrated in FIGS. 2 and 15, the distal ends of the accessory channels 16, 18 may be secured to the pivot arm 26. Therefore, the accessory channels 16, 18 may rotate with the pivot arm 26 when moving the pivot arm 26 between the side-viewing and forward-viewing configurations. FIG. 2 shows the accessory channels 16, 18 in the forward-viewing configuration, while FIG. 15 shows the accessory channels 16, 18 in the side-viewing configuration. As can be seen in FIG. 15, when in the side-viewing configuration and due to the rotation of the pivot arm 26, distal portions of the accessory channels 16, 18 are bent outside of the confines of the ribs 22 and then curve back towards and into the pivot arm 26. Thus, in the forward-viewing configuration, the angle of curvature or bending radius of the distal portion 12, or angle of distal portion curvature, is the same as the angle of curvature of the accessory channels 16, 18 such that the accessory channels 16, 18 and the distal portion 12 of the endoscope system 10 are substantially parallel; but in the side-viewing configuration, the angle of curvature or bending radius of the accessory channels 16, 18 is greater than the angle of curvature of the distal portion 12 such that distal portions of the accessory channels 16, 18 extend outside the at least one lumen 15 of the distal portion 12.

To move the pivot arm 26 from the forward-viewing configuration to the side-viewing configuration, the accessory channels 16, 18 may be pushed in a distal direction relative to proximal portion 13 and central portion 14, which applies a force through the accessory channels 16, 18 to the pivot arm 26. The resulting force causes the pivot arm 26 to rotate about the pivot point of the pin 34, thereby moving the accessory channels 16, 18 and pivot arm 26 into the side-viewing configuration. To move back to the forward-viewing configuration, a proximal force may be applied to the accessory channels 16, 18 relative to proximal portion 13 and central portion 14, thereby transferring the proximal force to the pivot arm 26. The proximal force then causes the pivot arm 26 to again rotate around the pivot point of the pin 34 in the opposite direction, thereby moving the accessory channels 16, 18 and the pivot arm 26 back to the forward-viewing configuration. To ensure that the accessory channels 16, 18 move in unison during these movements, the accessory channels 16, 18 may be secured together at any point along the length of the endoscope system 10, or even along the entire length. In one example, the accessory channels 16, 18 may be secured together using plastic tubing throughout the entire length of the central portion. In another example, the accessory channels 16, 18 may be secured together at the portions of the accessory channels 16, 18 that extend outside the constraints of the distal portion 12 when the endoscope system 10 is in the side-viewing configuration.

While a pivot arm 26 may be used to assist in transferring the accessory channels 16, 18 between forward-viewing and side-viewing configurations, a variety of other methods and structures may be used. Further, rather than using a pivot arm 26, multiple pivot arms may be used, or one pivot arm may be used for each accessory channel 16, 18. Therefore, each accessory channel 16, 18 may be moved between the forward-viewing and side-viewing configurations independently of each other. Further, the degree of rotation of the pivot arm 26 between the forward-viewing and side-viewing configuration may vary, potential ranging from 45 degrees to greater than 135 degrees.

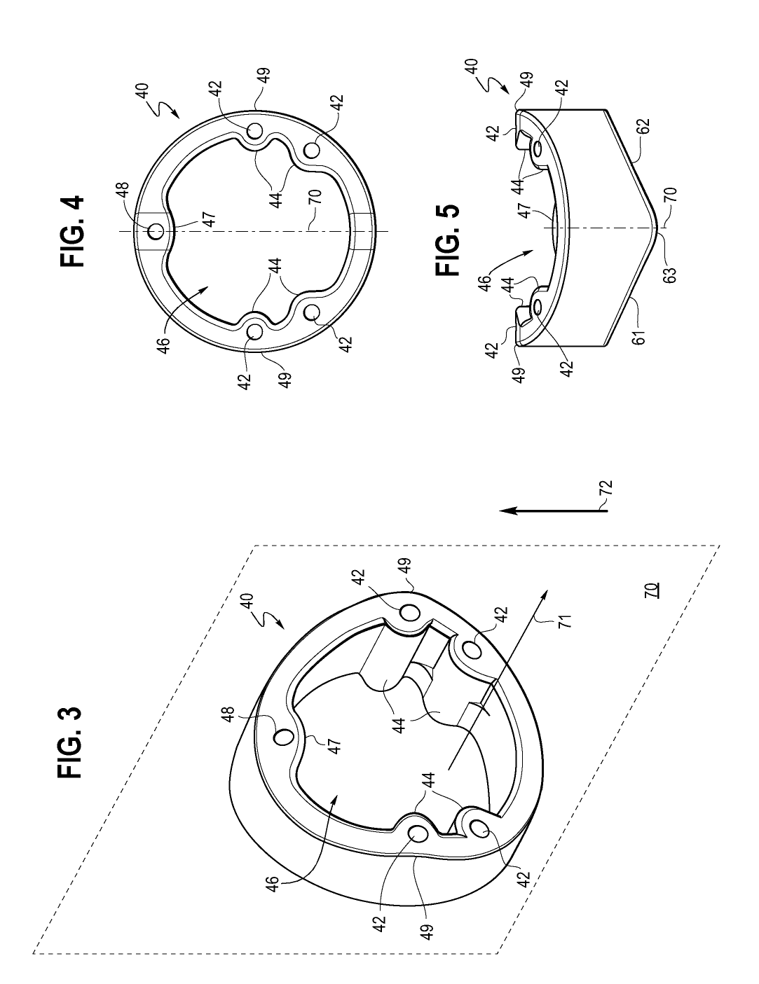

FIGS. 3-5 illustrate views of an example of a rib 22 for an example of an endoscope system 10 constructed according to the principles of the present disclosure, specifically a ring-shaped rib 40. Each ring-shaped rib 40 may be shaped to allow for minimal contact between the individual ring-shaped ribs 40. Endoscope system 10 may include one or more ring-shaped ribs 40 at the proximal end of the distal portion 12.

FIG. 3 illustrates a perspective view of an example of a circumferentially continuous, or “ring-shaped,” rib 40. Ring-shaped rib 40 may be symmetrical about a vertical plane 70 including both the longitudinal axis 71 and vertical axis 72 of endoscope system 10. Vertical axis 72 is perpendicular to longitudinal axis 71. Ring-shaped rib 40 may include an interior cavity 46 in which accessory channels 16, 18 may be disposed such that ring-shaped rib 40 encircles accessory channels 16, 18. Ring-shaped rib 40 may include one or more tab mounts integral to the ring-shaped rib 40 projecting toward the interior cavity 46 of the ring-shaped rib 40. The one or more tab mounts may extend the proximal-distal width of the ring-shaped rib 40. The one or more tab mounts may include a top tab mount 47. The one or more tab mounts may include a proximal-distal top tab mount lumen 48 therethrough. Top tab mount 47 may include top tab mount lumen 48 extending from the proximal end of the top tab mount 47 through to the distal end of the top tab mount 47. The one or more tab mounts may further include one or more side tab mounts 44 projecting toward the interior cavity 46 of the ring-shaped rib 40 from each side of the ring-shaped rib 40. In some examples of a ring-shaped rib 40, the one or more side tab mounts 44 are symmetrically positioned about the vertical plane 70. Each of the one or more side tab mounts 44 includes a proximal-distal side tab mount lumen 42 therethrough. In further examples of a ring-shaped rib 40, each side of the ring-shaped rib 40 may have one side tab mount 44. The ring-shaped rib 40 may include rib side surface points 49 that protrude longitudinally further than the rib top and rib bottom surfaces.

FIG. 4 illustrates a longitudinal proximal-distal cross-sectional view of an example of a ring-shaped rib 40 highlighting the symmetry of the sides of ring-shaped rib 40 about the vertical plane 70 including the longitudinal axis 71, top tab mount 47, and top tab mount lumen 48.

FIG. 5 illustrates a bottom view of an example of a ring-shaped rib 40 highlighting the symmetry of the sides of ring-shaped rib 40 about the vertical plane 70 including the longitudinal axis 71, top tab mount 47, and top tab mount lumen 48 (not shown in FIG. 5). FIG. 5 illustrates the asymmetry of the longitudinal surfaces of ring-shaped rib 40, one longitudinal surface of which will be the proximal surface, and one longitudinal surface of which will be the distal surface. Because ring-shaped ribs 40 will alternate in orientation longitudinally along a distal portion 12 of an endoscope system 10, the distal surface of one ring-shaped rib 40 will correspond to the proximal surface of the next ring-shaped rib 40. Such proximal surface of the next ring-shaped rib 40 will correspond to the distal surface of the subsequent sequential ring-shaped rib 40, and so forth. In a particular ring-shaped rib 40, rib side surface points 49 may be on one longitudinal surface, whether such one longitudinal surface faces the proximal end or the distal end of the endoscope system 10, and planar side longitudinal surfaces 61, 62 may be on the opposite longitudinal surface. Planar side longitudinal surfaces 61, 62 may intersect at intersection surface 63. Planar side longitudinal surface 61 may be co-planar to a plane that is at an acute angle (i.e., less than 90 degrees) relative to a plane that perpendicularly cross-sects the proximal-distal longitudinal axis 71, perpendicular to vertical plane 70. Planar side longitudinal surface 62 may be co-planar to a second plane that is at an acute angle relative to the plane that perpendicularly cross-sects the proximal-distal longitudinal axis 71. A ring-shaped rib 40 may generally have a 90 degree rotation-reflection symmetry about an axis parallel to, or the same as, the longitudinal axis 71, alternatively referred to by those of ordinary skill as an improper axis of rotation, and more particularly, an S₄ symmetry axis. In other words, each ring-shaped rib 40 may be generally symmetrical when rotated 90 degrees about the longitudinal axis, and reflected through the longitudinal axis 71, such that rib side surface points 49 may also be intersection surfaces of two planar longitudinal surfaces, each of which may be co-planar to a plane that is at an acute angle relative to the plane that perpendicularly cross-sects the proximal-distal longitudinal axis 71. Such rotation-reflection symmetry may be without respect to the positions of any tab mounts or tab mount holes, which may not be included in the rotation-reflection symmetry.

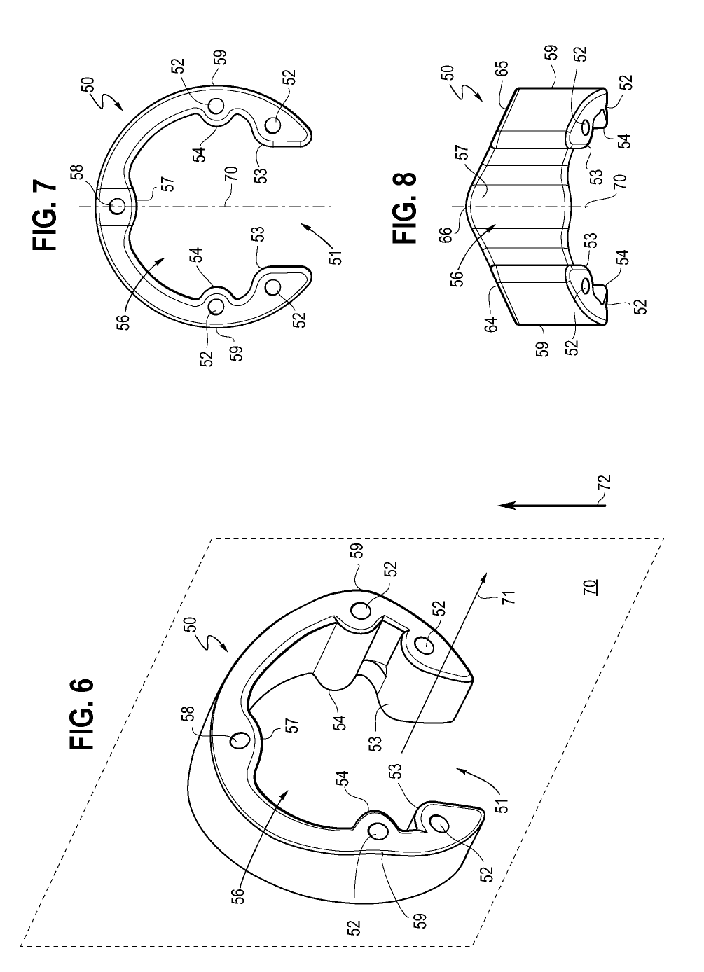

FIGS. 6-8 illustrate views of another example of a rib 22 for an example of an endoscope system 10 constructed according to the principles of the present disclosure, specifically a circumferentially uncontinuous rib 50, which may be generally “C-shaped” in some examples. Each circumferentially uncontinuous rib 50 may be shaped to allow for minimal contact between the individual circumferentially uncontinuous ribs 50. Endoscope system 10 may include one or more circumferentially uncontinuous ribs 50 in the distal portion 12 between the distal-most ring-shaped rib 40 and the distal end section 24.

FIG. 6 illustrates a perspective view of an example of a circumferentially uncontinuous rib 50. Circumferentially uncontinuous rib 50 may be symmetrical about a vertical plane 70 including the longitudinal axis 71 of endoscope system 10. Circumferentially uncontinuous rib 50 may include an interior cavity 56 in which accessory channels 16, 18 may be disposed such that circumferentially uncontinuous rib 50 generally surrounds accessory channels 16, 18 and accessory channels 16, 18 may be reversibly removed from interior cavity 56 by passing accessory channels 16, 18 through bottom rib opening 51. Bottom rib opening 51 interrupts the circumferential continuity of circumferentially uncontinuous rib 50. Circumferentially uncontinuous rib 50 may include one or more tab mounts integral to the circumferentially uncontinuous rib 50 projecting toward the interior cavity 56 of the circumferentially uncontinuous rib 50. The one or more tab mounts may extend the proximal-distal width of the circumferentially uncontinuous rib 50. The one or more tab mounts may include a top tab mount 57. The top tab mount 57 may include a proximal-distal top tab mount lumen 58 therethrough. Top tab mount lumen 58 extends from the proximal end of the top tab mount 57 through to the distal end of the top tab mount 57. The one or more tab mounts may further include rib opening-adjacent tab mounts 53, which may be flush with surfaces of the circumferentially uncontinuous rib 50 defining bottom rib opening 51. In some examples of a circumferentially uncontinuous rib 50, the rib opening-adjacent tab mounts 53 are symmetrically positioned about the vertical plane 70. Each of the rib opening-adjacent tab mounts 53 includes a proximal-distal side tab mount lumen 52 therethrough. In some examples of a circumferentially uncontinuous rib 50, the circumferentially uncontinuous rib 50 may further include one or more side tab mounts 54 positioned between each rib opening-adjacent tab mount 53 and the top tab mount 57. Each of the one or more side tab mounts 54 includes a proximal-distal side tab mount lumen 52 therethrough. The circumferentially uncontinuous rib 50 may include rib side surface points 59 that protrude longitudinally further than the rib top surface.

FIG. 7 illustrates a longitudinal proximal-distal cross-sectional view of an example of a circumferentially uncontinuous rib 50 highlighting the symmetry of the sides of circumferentially uncontinuous rib 50 about the vertical plane 70 including the longitudinal axis 71, top tab mount 57, and top tab mount lumen 58.

FIG. 8 illustrates a bottom view of an example of a circumferentially uncontinuous rib 50 highlighting the symmetry of the sides of circumferentially uncontinuous rib 50 about the vertical plane 70 including the longitudinal axis 71, top tab mount 57, and top tab mount lumen 58 (not shown in FIG. 8). FIG. 8 illustrates the asymmetry of the longitudinal surfaces of circumferentially uncontinuous rib 50, one longitudinal surface of which will be the proximal surface, and one longitudinal surface of which will be the distal surface. Because circumferentially uncontinuous ribs 50 will alternate in orientation longitudinally along a distal portion 12 of an endoscope system 10, the distal surface of one circumferentially uncontinuous rib 50 will correspond to the proximal surface of the next circumferentially uncontinuous rib 50. Such proximal surface of the next circumferentially uncontinuous rib 50 will correspond to the distal surface of the subsequent sequential circumferentially uncontinuous rib 50, and so forth. In a particular circumferentially uncontinuous rib 50, rib side surface points 59 may be on one longitudinal surface, whether such one longitudinal surface faces the proximal end or the distal end of the endoscope system 10, and planar side longitudinal surfaces 64, 65 may be on the opposite longitudinal surface. Planar side longitudinal surfaces 64, 65 may intersect at intersection surface 66. Planar side longitudinal surface 64 may be co-planar to a plane that is at an acute angle relative to a cross-sectional plane that perpendicularly cross-sects the proximal-distal longitudinal axis 71. Planar side longitudinal surface 65 may be co-planar to a second plane that is at an acute angle relative to the plane that perpendicularly cross-sects the proximal-distal longitudinal axis 71. A circumferentially uncontinuous rib 50 may generally have a 90 degree rotation-reflection symmetry about an axis parallel to, or the same as, the longitudinal axis 71, alternatively referred to as an improper axis of rotation, and more particularly, an S₄ symmetry axis. In other words, each circumferentially uncontinuous rib 50 may be generally symmetrical when rotated 90 degrees about the longitudinal axis 71, and reflected through the longitudinal axis 71, such that rib side surface points 59 may also be intersection surfaces of two planar longitudinal surfaces, each of which may be co-planar to a plane that is at an acute angle relative to the plane that perpendicularly cross-sects the proximal-distal longitudinal axis 71. Such rotation-reflection symmetry may be without respect to the positions of any tab mounts or tab mount holes, or bottom rib opening 51, which may not be included in the rotation-reflection symmetry.

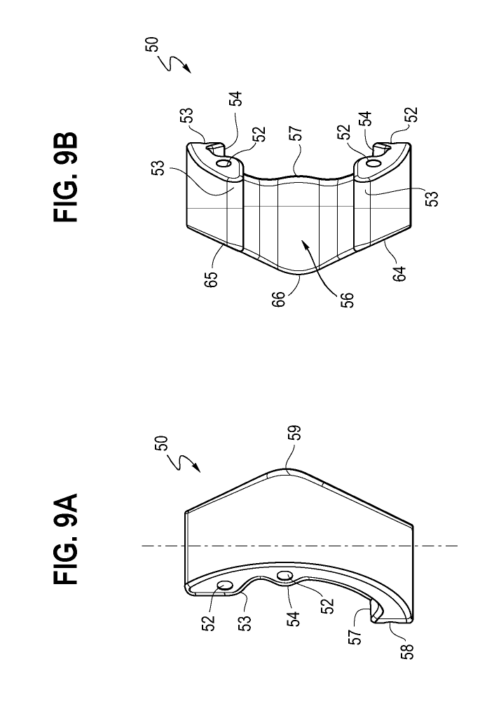

FIGS. 9A and 9B illustrate a view of the rotation-reflection symmetry associated with an example of a circumferentially uncontinuous rib 50 for an example of an endoscope system 10 constructed according to the principles of the present disclosure. FIG. 9A illustrates a side view of a circumferentially uncontinuous rib 50. As FIG. 9A demonstrates, rib side surface point 59 may be an intersection surface of two planar longitudinal surfaces. FIG. 9B illustrates a top view of a circumferentially uncontinuous rib 50, which corresponds to a 90-degree rotation-reflection about the longitudinal axis 71 of the circumferentially uncontinuous rib 50 illustrated in FIG. 9A. As FIG. 9B demonstrates, planar side longitudinal surfaces 64, 65 may intersect at intersection surfaces 66 similarly to two planar longitudinal surfaces intersecting at rib side surface point 59 in FIG. 9A. The 90-degree rotation-reflection symmetry of ring-shaped ribs 40 and circumferentially uncontinuous ribs 50 advantageously provide for four-way deflection of the bending section of the distal portion 12 of the endoscope system 10. The bending section may advantageously move exactly laterally, rather than at an angle. Further, such 90-degree rotation-reflection symmetry advantageously provides for bending of the bending section equally left (i.e., “a first direction”) and right (i.e., “a second direction,” opposite to the first direction) in the horizontal plane including longitudinal axis 71 and up (i.e., “a third direction,” perpendicular to the first direction and perpendicular to the second direction) and down (i.e., “a fourth direction,” opposite to the third direction) in the vertical plane 70.

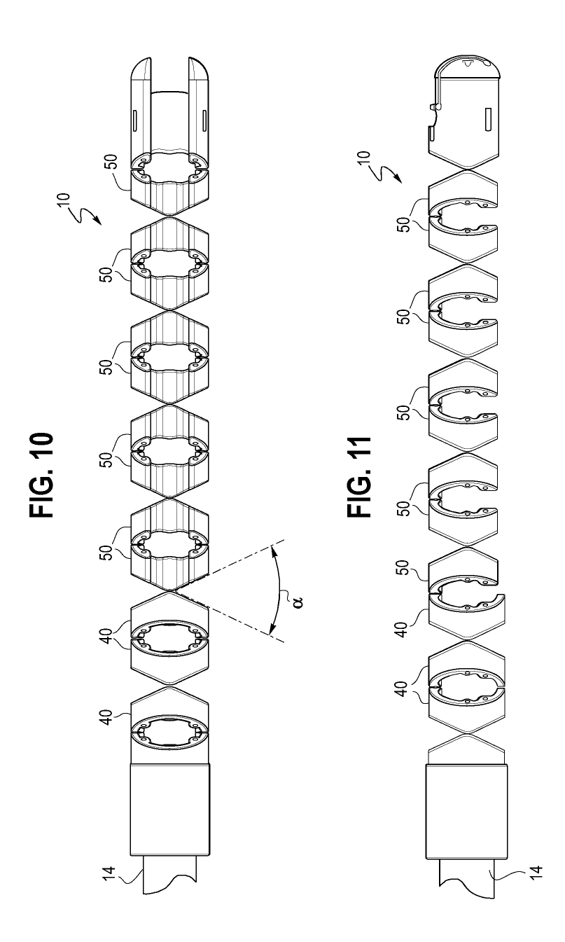

FIGS. 10 and 11 illustrate detailed views of an example of a distal portion 12 of an endoscope system 10 constructed in accordance with the principles of the present disclosure. FIG. 10 illustrates a top view of an example of a distal portion 12 of an endoscope system 10 constructed in accordance with the principles of the present disclosure. The distal portion 12 of endoscope system 10 may include one or more ring-shaped ribs 40 at the proximal end of the distal portion 12. The one or more ring-shaped ribs 40 are arranged sequentially in opposite orientations, with the proximal-most ring-shaped rib 40 arranged such that intersection surface 63 may be directed distally. The adjacent ring-shaped rib 40 to the proximal-most ring-shaped rib 40 is arranged such that the intersection surface 63 may be directed proximally to confront the intersection surface 63 of the proximal-most ring-shaped rib 40. The next distal ring-shaped rib 40 in sequence is arranged such that the intersection surface 63 may be directed distally. The bending section may include further distal ring-shaped ribs 40 in pairs, and such pairs may be arranged such that the intersection surface 63 of the distal-most ring-shaped rib 40 may be directed distally. Planar side longitudinal surface 61 of the proximal-most ring-shaped rib 40 and planar side longitudinal surface 62 of the adjacent ring-shaped rib 40 to the proximal-most ring-shaped rib 40 may form an angle. Depending upon left and right movement of the bending section during operation, the angle between a planar side longitudinal surface 61 directed proximally and a planar side longitudinal surface 62 of an adjacent ring-shaped rib 40 directed distally may be an angle from about 5 degrees to about 90 degrees, preferably from about 40 degrees to about 60 degrees.

The bending section will include one or more circumferentially uncontinuous ribs 50 distal to the distal-most ring-shaped rib 40. The proximal-most circumferentially uncontinuous rib 50 may be arranged such that intersection surface 66 may be directed proximally to confront the intersection surface 63 of the distal-most ring-shaped rib 40. Planar side longitudinal surface 61 of the distal-most ring-shaped rib 40 and planar side longitudinal surface 64 of the proximal-most circumferentially uncontinuous rib 50 may form an angle denoted in FIG. 10 by α. Depending upon left and right movement of the bending section during operation, the angle α may be an angle from about 5 degrees to about 90 degrees, preferably from about 40 degrees to about 60 degrees. The bending section may include further circumferentially uncontinuous ribs 50 in pairs, and such pairs may be arranged such that the intersection surface 66 of the distal-most circumferentially uncontinuous rib 50 may be directed proximally. Planar side longitudinal surface 64 of a circumferentially uncontinuous rib 50 directed distally and planar side longitudinal surface 65 of an adjacent circumferentially uncontinuous rib 50 directed proximally may form an angle. Depending upon left and right movement of the bending section during operation, the angle between planar side longitudinal surface 64 and adjacent planar side longitudinal surface 65 may be an angle from about 5 degrees to about 90 degrees, preferably from about 40 degrees to about 60 degrees.

FIG. 11 illustrates a side view of an example of a distal portion 12 of an endoscope system 10 constructed in accordance with the principles of the present disclosure. While the proximal-most ring-shaped rib 40 is arranged such that the intersection surface 63 may be directed distally, due to the advantageous 90-degree rotation-reflection symmetry of ring-shaped rib 40, the rib side surface point 49 may be directed proximally, as illustrated by FIG. 11. The two ring-shaped ribs 40 adjacent to the proximal-most ring-shaped rib 40 are arranged such that the rib side surface point 49 of one ring-shaped rib 40 confronts the rib side surface point 49 of the adjacent ring-shaped rib 40, as illustrated by FIG. 11. Planar side longitudinal surfaces of ring-shaped ribs 40 intersect at rib side surface point 49 such that adjacent ring-shaped ribs 40 that confront each other at their rib side surface points 49 may form an angle between planar side longitudinal surfaces of adjacent ring-shaped ribs 40. Depending upon up and down movement of the bending section during operation, the angle between planar side longitudinal surfaces may be an angle from about 5 degrees to about 90 degrees, preferably from about 40 degrees to about 60 degrees. Planar side longitudinal surfaces of circumferentially uncontinuous ribs 50 intersect at rib side surface points 59 such that adjacent circumferentially uncontinuous ribs 50 that confront at their rib side surface points 59 may form an angle between planar side longitudinal surfaces of adjacent circumferentially uncontinuous ribs 50. Depending upon up and down movement of the bending section during operation, the angle between planar side longitudinal surfaces may be an angle from about 5 degrees to about 90 degrees, preferably from about 40 degrees to about 60 degrees.

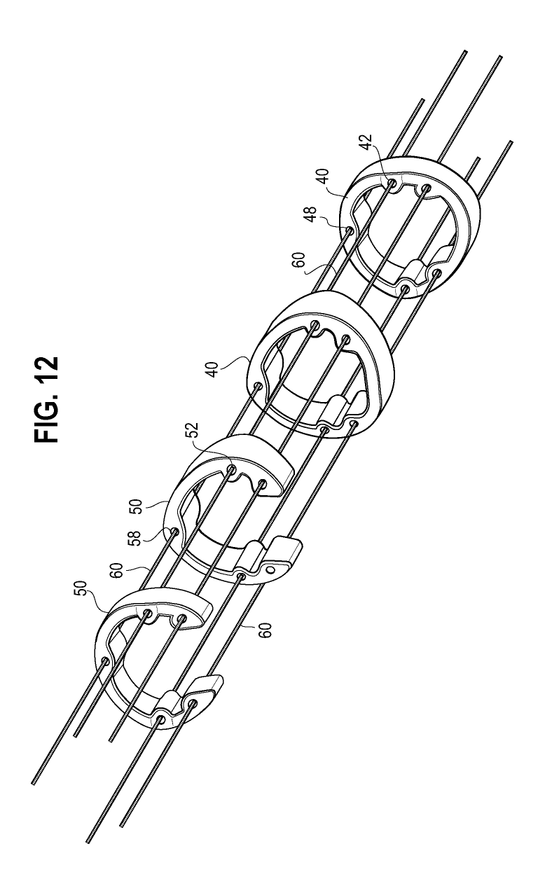

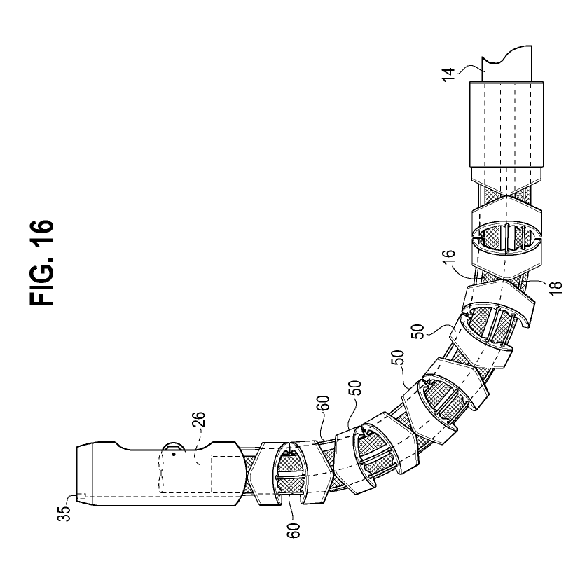

FIG. 12 illustrates another example of an assembly of an example of ribs 22 of on a series of one or more control wires 60. Control wires 60 may be fixedly attached to the distal end section 24 and extend in parallel through, or outside of the at least one lumen 15 to handle portion 13. Alternatively, the control wires 60 may extend through dedicated low friction lumens or catheters along the length of the endoscope system 10 to the handle portion 13. A first control wire 60 may be fixed on a wall of the distal end section 24 such that control wire 60 passes through top tab mount lumen 58 of one or more circumferentially uncontinuous ribs 50 and through top tab mount lumen 48 of one or more ring-shaped ribs 40. Additional control wires 60 may be fixed in advantageous positions on the wall of the distal end section 24 such that a control wire 60 passes through a side tab mount lumen 52 of the one or more circumferentially uncontinuous ribs 50 and through a corresponding side tab mount lumen 42 of the one or more ring-shaped ribs 40, as illustrated in FIG. 12. An endoscope system 10 may include three, four, five, or more control wires 60. In addition to the ability to switch between forward-viewing and side-viewing configurations, the distal portion 12 of the endoscope system 10 may also bend and rotate as desired advantageously. FIG. 15 illustrates the distal portion 12 in a straight configuration such that the control wires 60 are straight and parallel, while FIG. 16 illustrates the distal portion 12 in a bent configuration, such that the control wires 60 are parallel but not straight. To move the distal portion 12 from the straight configuration illustrated in FIG. 15 to the bent configuration illustrated in FIG. 16, a control wire 60 may be pulled in a proximal direction. The proximal movement of the control wire 60 may result in a force being applied through the control wire 60 and to the distal end section 24. This force may cause the flexible, ribbed body of the distal portion 12 to bend as illustrated in FIG. 15. To move the distal portion 12 back to the straight configuration, second and third control wires 60 opposite to the initially-pulled control wire 60 may be pulled in a proximal direction. Because the second and third control wires 60 are connected to the opposite side of the distal end section 24, a force is applied through the second and third control wires 60 and to the distal end section 24 that may move the distal portion 12 back towards the straight configuration. The side tab mount lumens 42 and 52 of the ring-shaped rib 40 and circumferentially uncontinuous rib 50, respectfully, may be advantageously arranged such that control wires 60 may allow for force to be applied through the control wires 60 and to the distal end section 24, and reversibly move the distal end section 24 up, down, left, or right. The side tab mount lumens 42 and 52 may be advantageously positioned on the one or more ring-shaped ribs 40 and the one or more circumferentially uncontinuous ribs 50 such that the bending section will not be pulled off-plane, which allows for the bending section to move advantageously exactly laterally rather than at an angle and which force transmission is not increased by pulling a ring-shaped rib 40 or circumferentially uncontinuous rib 50 at a moment. Circumferentially uncontinuous ribs 50 may be arranged on control wires 60 such that the bottom rib openings 51 may be aligned longitudinally, such that the bottom rib openings 51 are open to the same direction, or coaxial.

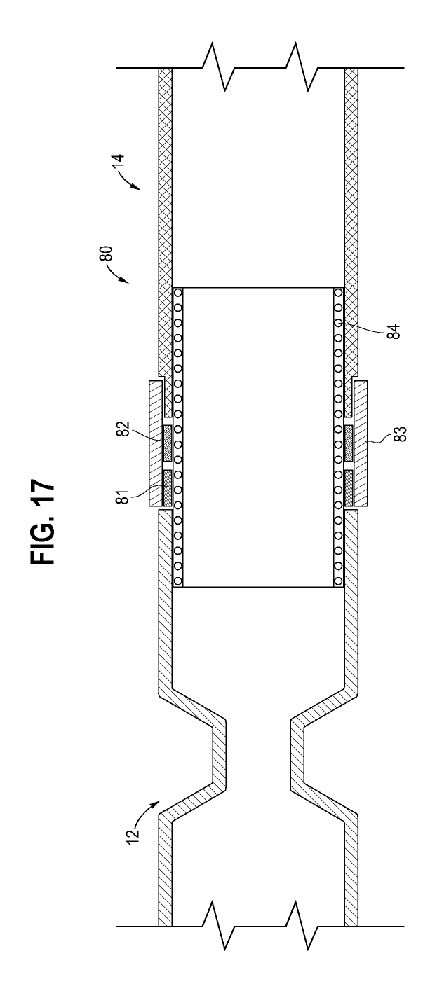

FIG. 17 illustrates a detailed view of an example of an axially rotatable bearing 80 of an example of an endoscope system 10 and its functionality. The axially rotatable bearing 80 may include a first ring 81 and a second ring 82. The axially rotatable bearing 80 may further include a first tube 83 and a second tube 84. The first tube 83 may be fixedly attached to the central portion 14 and the first ring 81. The second tube 84 may be fixedly attached to the distal portion 12 and the second ring 82. The first tube 83 and first ring 81 may be freely rotatable with respect to the second tube 84 and second ring 82, thereby making the distal portion 12 freely rotatable with respect to the central portion 14. Because the first ring 81 is indirectly secured to the central portion 14, but is located distal to the second ring 82, which is indirectly secured to the distal portion 12, the distal portion 12 and central portion 14 may remain secured to each other while still remaining freely rotatable with respect to each other. The distal portion 12 may be freely rotated when the endoscope system 10 is in any one of the configurations described above, including forward-viewing configuration, side-viewing configuration, straight configuration, and bent configuration. The accessory channels 16, 18 and the control wires 60 may pass freely through lumen 15 of the axially rotatable bearing 80 while causing no or minimal interference to the axially rotatable bearing 80. This is merely one potential design for the axially rotatable bearing 80, and various other designs that allow free rotation of the distal portion 12 with respect to the central portion 14 may be used.

Control wires 60 may also advantageously secure the one or more ring-shaped ribs 40 and the one or more circumferentially uncontinuous ribs 50 of the distal portion 12 together. Sufficient tension may be applied to the control wires 60, thereby advantageously securing together the one or more ring-shaped ribs 40 and the one or more circumferentially uncontinuous ribs 50 along the control wires 60. Due to this design, the one or more ring-shaped ribs 40 and the one or more circumferentially uncontinuous ribs 50 may be advantageously shaped to allow for minimal contact between the individual one or more ring-shaped ribs 40 and/or the one or more circumferentially uncontinuous ribs 50. Optionally, one or more control wires 60 may include built-in electrical wiring from an electrical power source that allows the one or more control wires 60 to function as a circuit for the LED light 35 as well. Alternatively, or in addition to the one or more control wires 60, the one or more ring-shaped ribs 40 and/or the one or more circumferentially uncontinuous ribs 50 may be connected together using a variety of other methods, such as with mechanical hinges, adhesives, and other well-known devices. Further, additional elongate members may extend through the top tab mount lumens 48 and 58 and/or side tab mount lumens 42 and 52 similarly to the one or more control wires 60 to provide additional support to the distal portion 12.

The endoscope system 10 may move between a bent configuration and a straight configuration while the endoscope system 10 may also be in either the forward-viewing or side-viewing configurations. For example, FIG. 15 illustrates that the endoscope system 10 in a straight and side-viewing configuration. The endoscope system 10 may be manipulated and used in any combination of the above-mentioned configurations, and may be repeatedly movable between all configurations.

The accessory channels 16, 18 may be used to provide access for a variety of medical tools and accessories through the endoscope system 10 and into a patient’s body. For example, a camera system may be inserted into one of the accessory channels 16, while a variety of tools including, but not limited to, forceps, sphincterotomes, wires, dilation balloons, extraction balloons, stents, needle knives, hemostasis clips, and any other catheter-based tool may be inserted into the other accessory channel 18. The tools may be advanced past the distal ends of the accessory channels 16, 18 where they may be used to operate on a patient.

The endoscope system 10, or any portion thereof, may be designed to be disposable, thus reducing the risk of bacterial infection due to incomplete cleaning between uses.

The present disclosure further contemplates methods of using an endoscope system 10, including the steps of: inserting the endoscope system 10 into a patient’s body, the endoscope system 10 including an elongate tube including at least one lumen 15 and one or more accessory channels 16, 18 movably disposed at least partially within the at least one lumen 15 of the elongate tube, the one or more accessory channels 16, 18 including a tubular structure including first and second accessory lumens 28, 30 extending therethrough; and moving a control wire 60 so as to bend the distal portion 12 of the elongate tube in a first direction.

A method of using an endoscope system 10 may further include the step of: positioning the endoscope system 10 in a forward-viewing configuration, wherein in the forward-viewing configuration the distal end section 24 of the one or more accessory channels 16, 18 is substantially parallel to the distal portion 12 of the elongate tube.

A method of using an endoscope system 10 may further include the step of: moving the endoscope system 10 to a side-viewing configuration wherein in the side-viewing configuration, the distal end section 24 of the one or more accessory channels 16, 18 is arced at a radius greater than a radius of the distal portion 12 of the elongate tube. The step of moving the endoscope system 10 to a side-viewing configuration may further include rotating the distal end section 24 of the one or more accessory channels 16, 18 about a pivot point of the distal portion 12 of the elongate tube.

A method of using an endoscope system 10 may further include the step of: moving a second control wire 60 so as to bend the distal portion 12 of the elongate tube in a second direction, the second direction opposite to the first direction.

A method of using an endoscope system 10 may further include the step of: moving a third control wire 60 so as to bend the distal portion 12 of the elongate tube in a third direction, the third direction perpendicular to the first direction and perpendicular to the second direction.

A method of using an endoscope system 10 may further include the step of: moving a fourth control wire 60 so as to bend the distal portion 12 of the elongate tube in a fourth direction, the fourth direction opposite to the third direction.

A method of using an endoscope system 10 may further include the step of: moving the endoscope system 10 from the side-viewing configuration back to the forward-viewing configuration.

A method of using an endoscope system 10 may further include the step of: removing the one or more accessory channels 16, 18 from the interior cavity 56 of the one or more circumferentially uncontinuous ribs 50.

### DISTAL END COVERINGS FOR BENDABLE ENDOSCOPE SYSTEMS

As described below, various coverings may be applied to the distal portion 12 of the exemplary bendable endoscope systems 10 of the present disclosure. These coverings may prevent the theoretical risk of tissue (e.g., within the gastrointestinal tract) from entering between the ribs 40, 50 within the distal portion 12 during use of the endoscope system 10. These coverings may also prevent the theoretical risk of tissue trauma from excessive rubbing of the exposed ribs 40, 50.

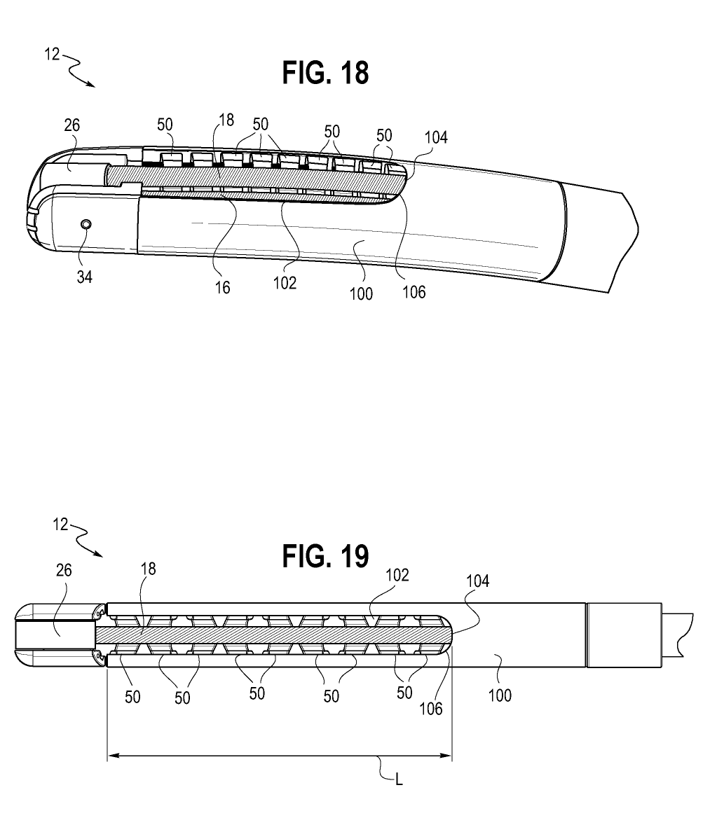

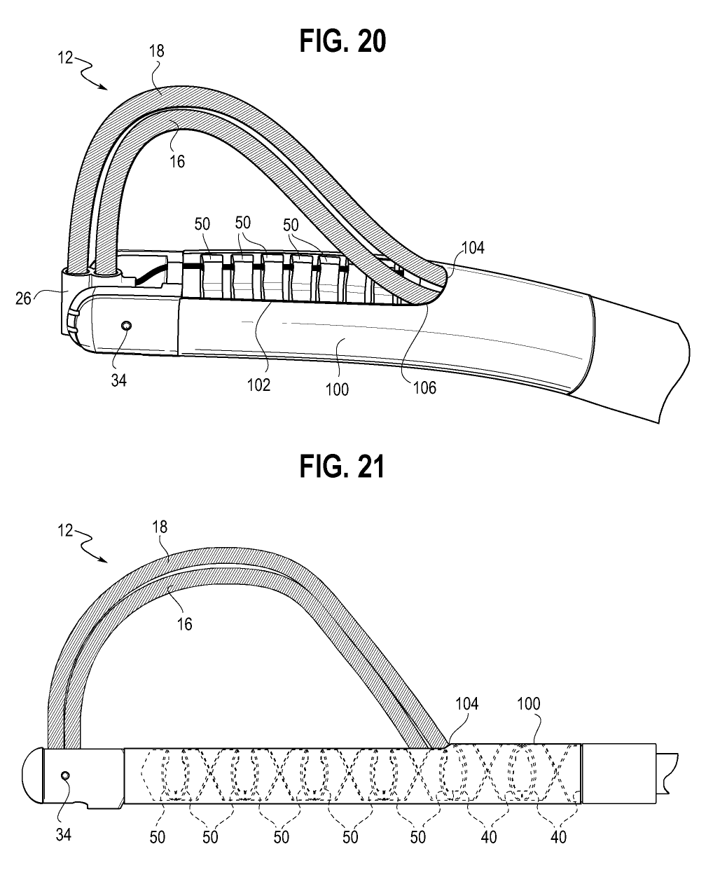

FIGS. 18 to 21 illustrate a first example of a distal portion 12 of an endoscope system constructed in accordance with the principles of the present disclosure, having a sleeve 100 surrounding at least a portion of the distal portion 12, and one or more of the ribs 40, 50 positioned therein. As seen best in FIGS. 18 to 20, the sleeve 100 may have a longitudinal slot 102 that substantially corresponds to the rib openings 51 of the circumferentially uncontinuous ribs 50, thereby enabling the accessory channels 16, 18 to move between positions within the interior cavities 56 of the circumferentially uncontinuous ribs 50 (e.g., when in a forward-viewing configuration), as seen in FIGS. 18 to 19, through the longitudinal slot 102, to positions outside the interior cavities 56 of the circumferentially uncontinuous ribs 50 (e.g., when in a side-viewing configuration), as seen in FIGS. 20 to 21. In this way, the sleeve 100 operates as a protective barrier that limits interaction between the ribs 40, 50 and surrounding tissue, while the longitudinal slot 102 facilitates free movement of the accessory channels 16, 18 in to and out of the interior cavities 56 of the circumferentially uncontinuous ribs 50.

In some embodiments, the sleeve 100 may extend the entire longitudinal length of the distal end 12 (including some or all of the distal end section 24) , less than the entire length of the distal end 12, or greater than the entire length of the distal end 12, for example, into the central portion 14. Likewise, the length of the sleeve 100 may surround some or all of the circumferentially uncontinuous ribs 50, without covering any of the ring-shaped ribs 40, or the length of the sleeve 100 may surround some or all of the circumferentially uncontinuous ribs 50, along with one or more of the ring-shaped ribs 40.

The material of the sleeve 100 may comprise, in whole or in part, a fluoroelastomer, a synthetic rubber, a fluorocarbon rubber, a synthetic elastomer, a natural polymer, a hydrocarbon polymer, or composite material, such as Viton, Latex, Polyisoprene, Polyurethan, Silicone rubber, Nusil Silicon, TPE, natural rubber, or the like. The sleeve 100 may have a thickness, for example, ranging between 0.2 to 0.6 mm. In some embodiments, anti-sticking, chlorination, and curing surface treatments may be applied to the outer surface of the sleeve 100.

Those skilled in the art will appreciate that the dimensions and/or shape of the longitudinal slot 102 may affect the extent to which the accessory channels 16, 18 can move between forward-viewing configurations and side-viewing configurations, and/or the amount of curvature the accessory channels 16, 18 can obtain when transitioning to a side-viewing configuration from a forward-viewing configuration, as seen in FIGS. 20-21. In some embodiments, a width of the longitudinal slot 102 is greater than or equal to an outer diameter of the accessory channels 16, 18 and/or greater than or equal to a width of the rib openings 51 of the circumferentially uncontinuous ribs 50. For example, the width may range between 5 to 7 mm.

In some embodiments, the length of the longitudinal slot 102 (e.g., along the longitudinal axis of the endoscope system 10) may extend a length spanning at least two or more of the circumferentially uncontinuous ribs 50, and preferably, at least five of the circumferentially uncontinuous ribs 50. In some embodiments, the length of the longitudinal slot 102 may extend a length spanning at least 50% of the total number of the circumferentially uncontinuous ribs 50 in the distal portion 12, and preferably, at least 80% to 100% of the total number of the circumferentially uncontinuous ribs 50 in the distal portion 12. In some embodiments, the length L in FIG. 19 of the longitudinal slot 102 may range between 5 to 50 mm, and preferably 5 to 20 mm. In one embodiment, the length of the slot is about 10 mm.

In some embodiments, a proximal end 104 of the longitudinal slot 102 may comprise a curved and/or generally semi-circular shape 106 that approximates or matches the curvature or shape of accessory channels 16, 18. In addition to providing a surface against which the accessory channel 18 engages the sleeve 100 in a side-viewing configuration, the curved and/or generally semi-circular shape 106 may help prevent tearing of the covering when restricting advancement of the accessory channels 16, 18 out of the interior cavities 56 of the circumferentially uncontinuous ribs 50, for example, by limiting or otherwise distributing stress concentrations resulting from engagement of the accessory channels 16, 18 with the proximal end 104 of the longitudinal slot 102.

In some embodiments, a lubricant may be added to the inside surface of the sleeve 100 and/or the outer surface of the ribs 40, 50 to reduce frictional forces between the sleeve 100, the ribs 40, 50, and/or any components moving through the distal portion 12, such as the accessory channels 16, 18. One suitable lubricant is Dow Corning 360 Medical Fluid.

In some embodiments, the sleeve 100 may be fixed or adhered to the distal portion 12, for example, at one or more ribs 40, 50, by the application of a primer (such as Loctite Primer 7701 or similar) to an inner surface of the sleeve 100 and/or the outer surface of the one or more ribs 40, 50, followed by the application of cyanoacrylate (such as Loctite 4061 or similar). The fixation or adherence may occur at the proximal and distal ends of the sleeve 100, and/or at multiple locations along the longitudinal length of the sleeve 100. Additionally, or alternatively, two or more UV glue rings may be used at multiple locations (e.g., at the proximal and distal ends) along the sleeve 100 to further secure the sleeve 100 to the distal portion 12.

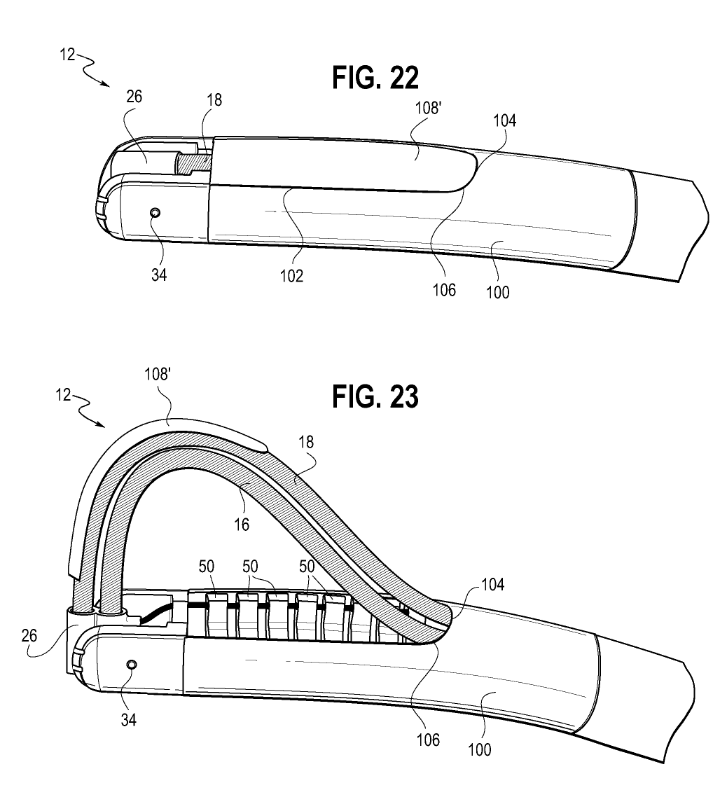

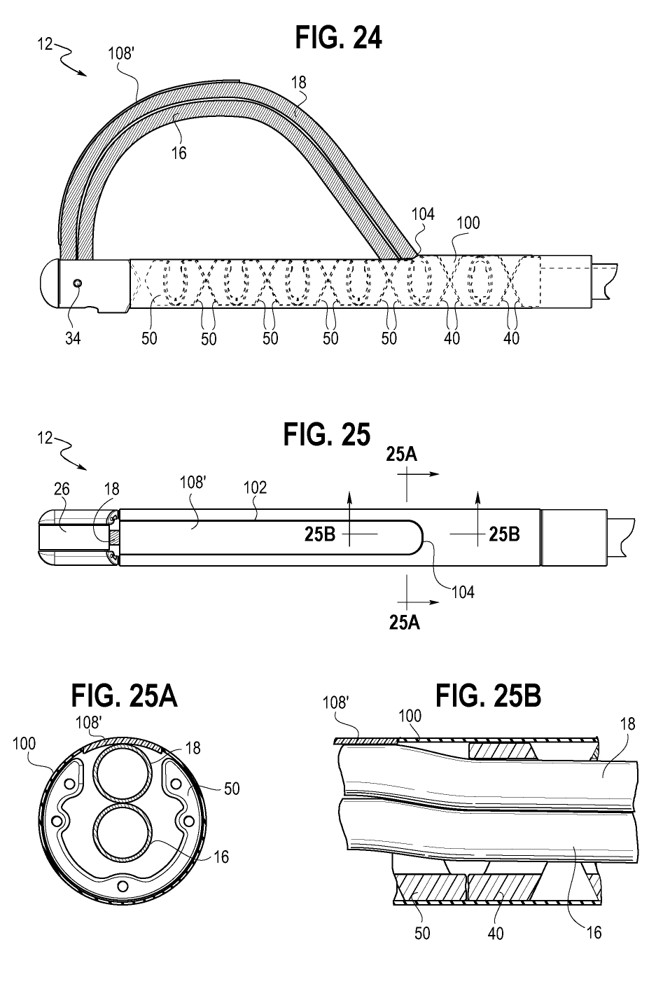

FIGS. 22 to 25, including FIGS. 25A-B, illustrate a second example of a distal portion 12 of an endoscope system constructed in accordance with the principles of the present disclosure, having a sleeve 100 and a patch 108’ surrounding the distal portion 12, and one or more of the ribs 40, 50 positioned therein. As compared to the example of FIGS. 18 to 21, the example of FIGS. 22 to 25 materially differs only by the addition of the patch 108’ fixed to and moveable with the accessory channel 18. The description of the example of FIGS. 18 to 21 otherwise applies equally to the example of FIGS. 22 to 25, including FIGS. 25A-B.

In this way, the patch 108’ in combination with the sleeve 100 form a full circumferential protective barrier about the distal portion 12 that limits interaction between the ribs 40, 50, the accessory channels 16, 18, and surrounding tissue when the accessory channels 16, 18 are within the interior cavities 56 of the circumferentially uncontinuous ribs 50 (e.g., in a forward viewing configuration), while the longitudinal slot 102 continues to facilitate free movement of the accessory channels 16, 18 in and out of the interior cavities 56 of the circumferentially uncontinuous ribs 50. Likewise, when the accessory channels 16, 18 are moved outside of the interior cavities 56 of the circumferentially uncontinuous ribs 50 (e.g., in a side viewing configuration), the patch 108’ continues to serve as a protective barrier between the accessory channels 16, 18, and surrounding tissue adjacent the patch 108’.

In some embodiments, the patch 108’ is sized and shaped to generally match or approximate the size and shape of the slot 102 formed in the sleeve 100, such that when the accessory channels 16, 18 are positioned within the interior cavities 56 of the circumferentially uncontinuous ribs 50 (e.g., in a forward viewing configuration), the patch 108’ sits within the slot 102 and flush with an external surface of the sleeve 100, as best illustrated in FIGS. 25A-B. This flush arrangement may aid in the movement of the endoscope system through a patient’s anatomy by maintaining a smooth external surface, while minimizing potential pinching or rubbing of surrounding tissues.

In some embodiments, a width of the patch 108’ is greater than or equal to an outer diameter of the accessory channels 16, 18 and/or greater than or equal to a width of the rib openings 51 of the circumferentially uncontinuous ribs 50. For example, the width may range between 5 to 7 mm. In some embodiments, the length of the patch 108’ (e.g., along the longitudinal axis of the endoscope system 10) may extend a length spanning at least two or more of the circumferentially uncontinuous ribs 50, and preferably, at least five of the circumferentially uncontinuous ribs 50. In some embodiments, the length of the patch 108’ may extend a length spanning at least 50% of the total number of the circumferentially uncontinuous ribs 50 in the distal portion 12, and preferably, at least 80% to 100% of the total number of the circumferentially uncontinuous ribs 50 in the distal portion 12. In some embodiments, the length of the patch 108’ may range between 5 to 50 mm, and preferably 5 to 20 mm. In one embodiment, the length of the patch 108’ is about 10 mm.

In some embodiments, the material of the patch 108’ may be the same as the material of the sleeve 100. Similarly, the thickness of the patch 108’ may be the same as the thickness of the sleeve 100. Likewise, anti-sticking, chlorination, and curing surface treatments may be applied to the outer surface of the patch 108’.

In some embodiments, the patch 108’ may be fixed or adhered to the accessory channel 18 in the same or similar manner that the sleeve 100 is fixed or adhered to the distal portion 12, for example, at one or more ribs 40, 50.

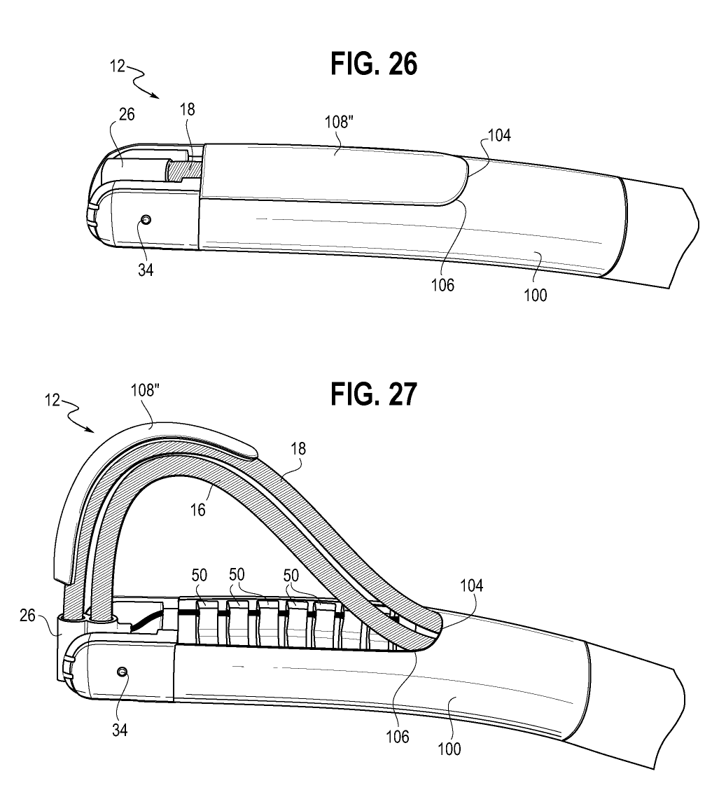

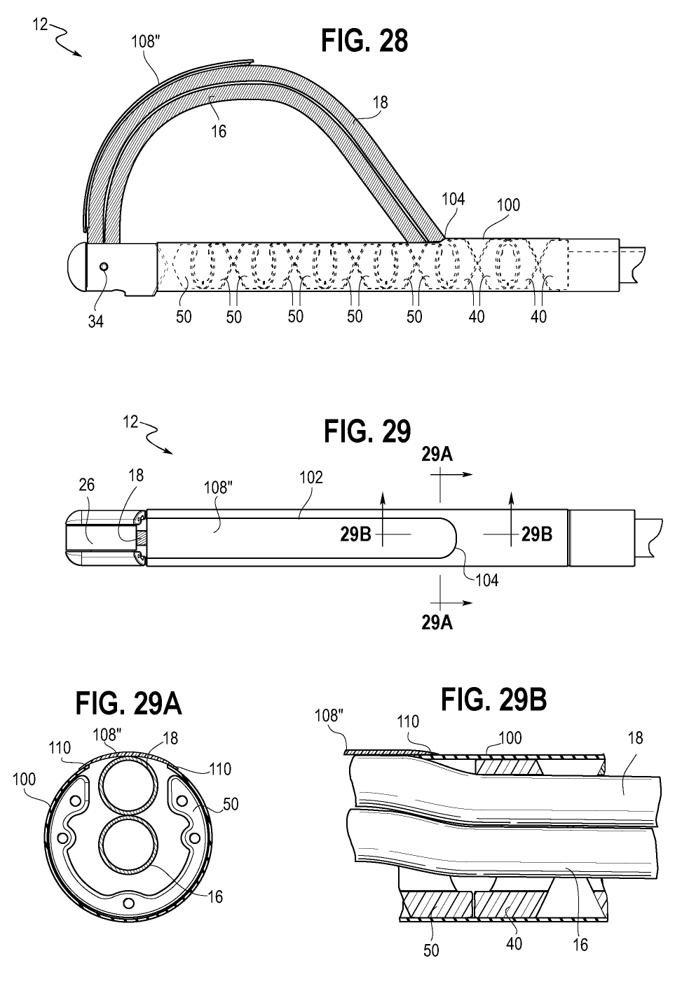

FIGS. 26 to 29, including FIGS. 29A-B, illustrate a third example of a distal portion 12 of an endoscope system constructed in accordance with the principles of the present disclosure, having a sleeve 100 and a patch 108’’ surrounding the distal portion 12, and one or more of the ribs 40, 50 positioned therein. As compared to the example of FIGS. 22 to 25, the example of FIGS. 26 to 29 materially differs only in that the patch 108’’ is sized and shaped to exceed the size and shape of the slot 102 formed in the sleeve 100, at least in one or more dimensions, such that when the accessory channels 16, 18 are positioned within the interior cavities 56 of the circumferentially uncontinuous ribs 50 (e.g., in a forward viewing configuration), at least a portion 110 of the patch 108’’ overlaps with an external surface of the sleeve 100, as best illustrated in FIGS. 29A-B. The description of the example of FIGS. 18 to 21 otherwise applies equally to the example of FIGS. 26 to 29, including FIGS. 29A-B.

In some embodiments, the overlapping portion 110 may occur along one or both of the longitudinal edges of the slot 102, as best illustrated in FIG. 29A, or it may occur along the proximal end 104 of the slot 102, as best illustrated in FIG. 29B. Alternatively, the overlapping portion 110 may occur along both the longitudinal edges of the slot 102, as well as at the proximal end 104 of the slot 102. The patch 108’’ may otherwise be fixed or adhered to the accessory channel 18 in the same manner as described above with regard to the patch 108’.

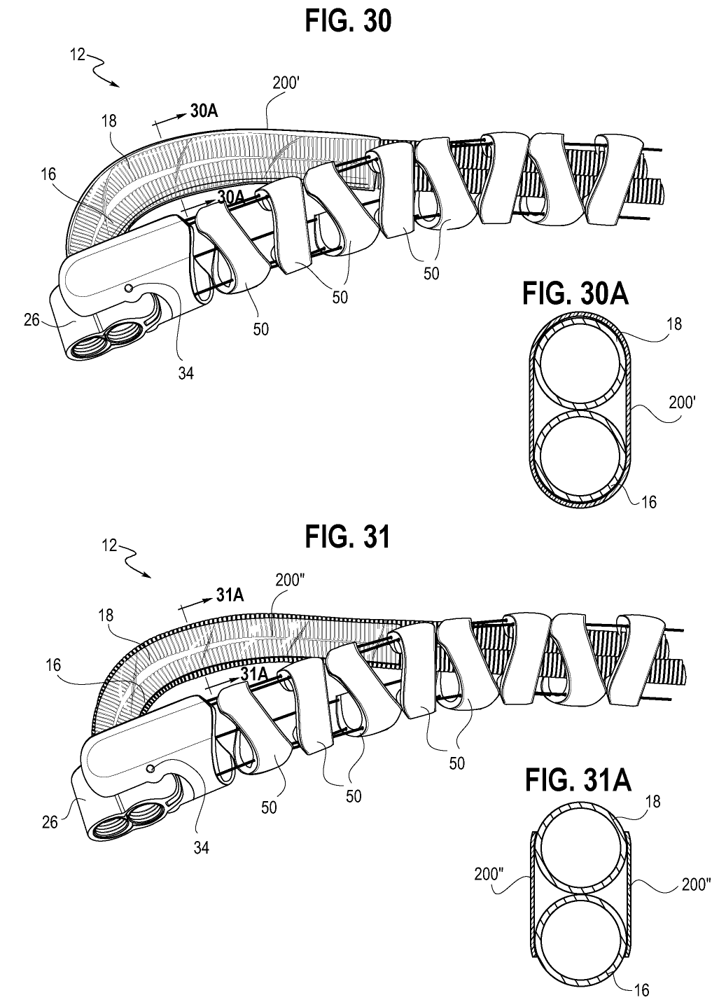

FIGS. 30 and 30A illustrate a fourth example of a distal portion 12 of an endoscope system constructed in accordance with the principles of the present disclosure, having an inner cover 200’ surrounding at least a portion of the accessory channels 16, 18. As illustrated, the inner cover 200’ is tubular, having a generally circular or elliptical shape, which completely surrounds the accessory channels 16, 18 about a periphery thereof, as well as any other structure (not shown) in the distal portion 12 connecting the accessory channels 16, 18 to one another. The inner cover 200’ therefore is movable with the accessory channels 16, 18 into and out of the interior cavities 56 of the circumferentially uncontinuous ribs 50.

In this way, the internal cover 200’ forms a full circumferential protective barrier around the accessory channels 16, 18 that limits interaction between the accessory channels 16, 18 and the circumferentially uncontinuous ribs 50 when the accessory channels 16, 18 are within, or moving into our out of, the interior cavities 56 of the circumferentially uncontinuous ribs 50 (e.g., in a forward viewing configuration). Likewise, when the accessory channels 16, 18 are moved outside of the interior cavities 56 of the circumferentially uncontinuous ribs 50 (e.g., in a side viewing configuration), the inner cover 200’ serves as a protective barrier between the accessory channels 16, 18 and surrounding tissue, as well as the circumferentially uncontinuous ribs 50, particularly on reentry of the accessory channels 16, 18 into the interior cavities 56 of the circumferentially uncontinuous ribs 50.

In some embodiments, a material of the inner cover 200’ may comprise, in whole or in part, a fluoroelastomer, a synthetic rubber, a fluorocarbon rubber, a synthetic elastomer, a natural polymer, a hydrocarbon polymer, or a composite material, such as Viton, Latex, Polyisoprene, Polyurethan, Silicone rubber, Nusil Silicon, TPE, natural rubber, or the like. The inner cover 200’ may have a thickness between 0.1 to 0.4 mm.

In some embodiments, the length of the inner cover 200’ (e.g., along the longitudinal axis of the endoscope system 10) may extend a length spanning at least two or more of the circumferentially uncontinuous ribs 50, and preferably, at least five of the circumferentially uncontinuous ribs 50. In some embodiments, the length of the inner cover 200’ may extend a length spanning at least 50% of the total number of the circumferentially uncontinuous ribs 50 in the distal portion 12, and preferably, at least 80% to 100% of the total number of the circumferentially uncontinuous ribs 50 in the distal portion 12. In some embodiments, the inner cover 200’ may have a length between 30 to 100 mm.

While the example of FIG. 30 is illustrated without the sleeve 100, those skilled in the art will appreciate that a distal portion 12 of an endoscope system constructed in accordance with the principles of the present disclosure may have both the inner cover 200’ and the sleeve 100. In such embodiments, the length of the inner cover 200’ may be greater than or equal to the length of the slot 102.

FIGS. 31 and 31A illustrate a fifth example of a distal portion 12 of an endoscope system constructed in accordance with the principles of the present disclosure, having an inner cover 200’’ surrounding at least a portion of the accessory channels 16, 18. As compared to the example of FIGS. 30 and 30A, the example of FIGS. 31 and 31A materially differs only in that the inner cover 200’’ only partially covers the accessory channels 16, 18. That is, the inner cover 200’’ may only cover the space or opening between the accessory channels 16, 18 and/or any other structure (not shown) in the distal portion 12 connecting the accessory channels 16, 18 to one another. In this way, the inner cover 200” may provide a protective barrier between surrounding tissues and the accessory channels 16, 18 (and/or any other structure in the distal portion 12 connecting the accessory channels 16, 18 to one another).

While the example of FIG. 31 (like the example of FIG. 30) is illustrated without the sleeve 100, those skilled in the art will appreciate that a distal portion 12 of an endoscope system constructed in accordance with the principles of the present disclosure may have both the inner cover 200’’ and the sleeve 100.

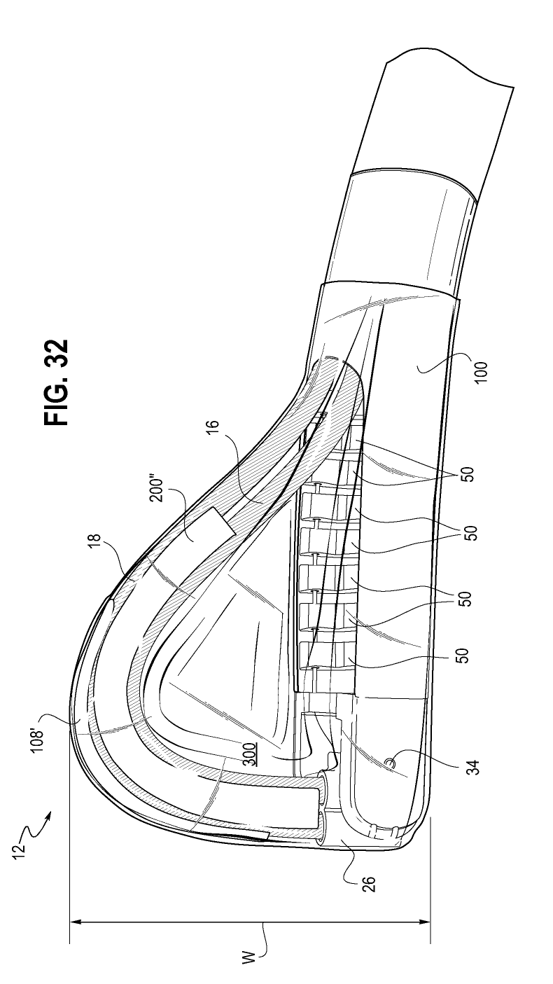

FIG. 32 illustrates a sixth example of a distal portion 12 of an endoscope system constructed in accordance with the principles of the present disclosure, having an elastic sleeve 300 surrounding at least a portion of the distal portion 12. The elastic sleeve 300 may be tubular, having a generally circular shape, which completely surrounds the distal portion 12 about a periphery thereof, including the circumferentially uncontinuous ribs 50, the rib openings 51, and the accessory channels 16, 18 (and any other sleeves or coverings therein, as discussed below). As illustrated, the elastic sleeve 300 is elastically deformable so as to permit the accessory channels 16, 18 to move between forward-viewing configurations and side-viewing configurations, while maintaining a protective barrier completely surrounding the periphery of the distal portion 12, even when the accessory channels 16, 18 are positioned outside of the interior cavities 56 of the circumferentially uncontinuous ribs 50. In this way, the elastic sleeve 300 helps reduce the risk of trauma by covering potentially rough or sharp parts of the distal portion 12, and also restricts the shape and height of the arc formed by the accessory channels 16, 18 in a side-viewing configuration. The elastic sleeve 300 further covers any pinch points of the “eye-shaped” gap formed between the accessory channels 16, 18 and the circumferentially uncontinuous ribs 50. Additionally, the elastic sleeve 300 prevents particles (for example, extracted bile stones) from impeding the accessory channels 16, 18 from re-entering the interior cavities 56 of the circumferentially uncontinuous ribs 50. Moreover, the elastic sleeve 300 may advantageously control the curvature of the accessory channels 16, 18, thereby limiting the overall width of the distal end portion 12 when the accessory channels 16, 18 are positioned outside of the interior cavities 56 of the circumferentially uncontinuous ribs 50. In some embodiments the width W in FIG. 32 may be between 15 to 35 mm. In one embodiment, the width W is about 25 mm.

In some embodiments, a material of the elastic sleeve 300 comprises, in whole or in part, a fluoroelastomer, a synthetic rubber, a fluorocarbon rubber, a synthetic elastomer, a natural polymer, a hydrocarbon polymer, or a composite material, such as Viton, Latex, Polyisoprene, Polyurethan, Silicone rubber, Nusil Silicon, TPE, natural rubber, or the like. In some embodiments, the elastic sleeve 300 may have a thickness between 0.1 to 0.4 mm, and a length between 30 to 110 mm. In some embodiments, the elastic sleeve 300 will have an expansion ability of at least 3, preferably 5, times the original unstretched diameter of the elastic sleeve 300.

In some embodiments, only a proximal and distal end of the elastic sleeve 300 may be fixed or adhered to the distal portion 12, so as to enable the elastic sleeve 300 to freely stretch and extend with the movement of the accessory channels 16, 18 out of and back into the interior cavities 56 of the circumferentially uncontinuous ribs 50. In some embodiments, the sleeve 300 may be fixed or adhered to the distal portion 12 in the same or similar manner that the sleeve 100 is fixed or adhered to the distal portion 12.

In some embodiments, an inner surface of the elastic sleeve 300 may be coated with a lubricant, such as Dow Corning 360 Medical Fluid, or similar, to reduce frictional forces between the elastic sleeve 300, ribs 40, 50, and/or the accessory channels 16, 18.

As illustrated in FIG. 32, a distal portion 12 of an endoscope system constructed in accordance with the principles of the present disclosure may include the elastic sleeve 300, the sleeve 100 with longitudinal slot 102, the patch 108’ (or the patch 108’’), and/or the inner cover 200’ (or the inner cover 200’’). It is envisioned that a distal portion 12 of an endoscope system constructed in accordance with the principles of the present disclosure may include any one, or a combination of, the elastic sleeve 300, the sleeve 100 with longitudinal slot 102, the patch 108’ (or the patch 108’’), and the inner cover 200’ (or the inner cover 200’’). To be clear, for example, a distal portion 12 of an endoscope system constructed in accordance with the principles of the present disclosure may include the elastic sleeve 300, with or without one or more of the sleeve 100 with longitudinal slot 102, the patch 108’ (or the patch 108’’), and/or the inner cover 200’ (or the inner cover 200’’).

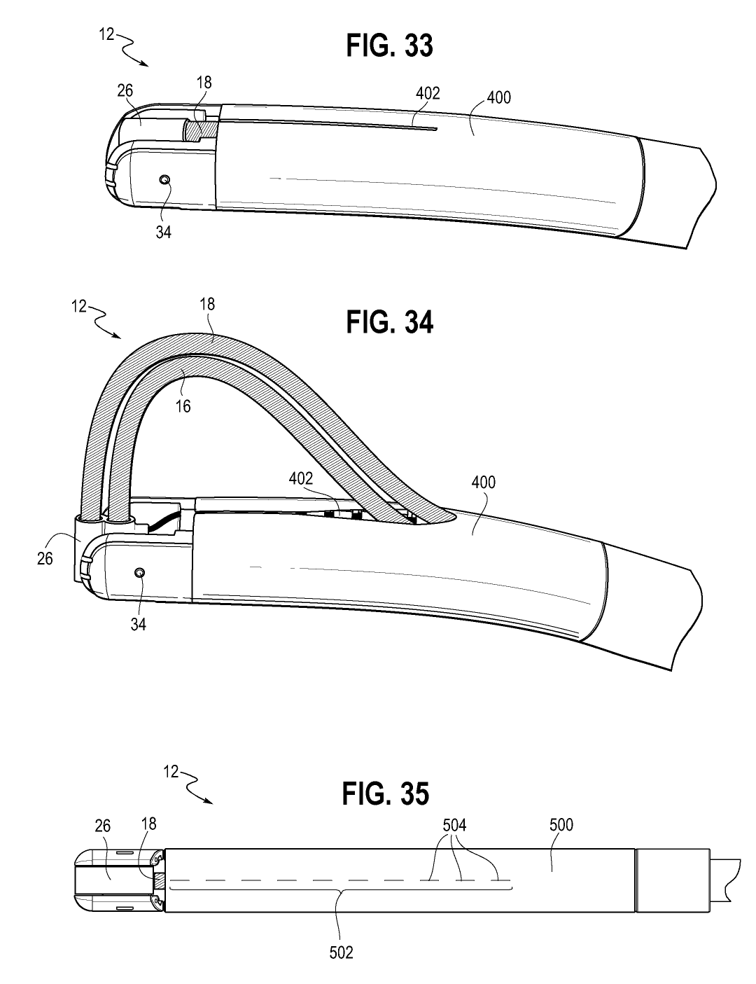

FIGS. 33 to 34 illustrates a seventh example of a distal portion 12 of an endoscope system constructed in accordance with the principles of the present disclosure, having a sleeve 400 surrounding at least a portion of the distal portion 12. As compared to the example of FIGS. 18 to 21, the only material difference in the example of FIGS. 33 to 34 is that a continuous longitudinal slit 402 (corresponding to the rib openings 51 of the circumferentially uncontinuous ribs 50) replaces the longitudinal slot 102. The longitudinal slit 402 differs from the longitudinal slot 102 in that the longitudinal slit 402 is dimensioned so that longitudinal edges of the slit 402 are either in contact with one another, or are nearly in contact with one another (e.g., separated by 0.5 mm, or less), such that the sleeve 400 entirely or at least substantially surrounds the circumference of the distal portion 12, while the longitudinal slit 402 facilitates movement of the accessory channels 16, 18 in to and out of the interior cavities 56 of the circumferentially uncontinuous ribs 50. Notably, due to the elastic nature of the sleeve 400, the slit 402 at least partially closes around the accessory channels 16, 18 when the accessory channels 16, 18 are positioned outside of the interior cavities 56 of the circumferentially uncontinuous ribs 50, thereby providing a protective barrier between the ribs 40, 50 positioned therein and surrounding tissues.

FIG. 35 illustrates an eighth example of a distal portion 12 of an endoscope system constructed in accordance with the principles of the present disclosure, having a sleeve 500 surrounding at least a portion thereof. As compared to the example of FIGS. 33 to 34, the only material difference in the example of FIG. 35 is that a longitudinal zone of weakness 502 (corresponding to the rib openings 51 of the circumferentially uncontinuous ribs 50) replaces the continuous longitudinal slit 402. The longitudinal zone of weakness 502 may comprise, for example, the continuous longitudinal slit 402, a plurality of individual perforations or notches 504, a thinning or reduction in the thickness of the sleeve 500, and/or other means for weakening the structural properties of the sleeve 500 within the longitudinal zone of weakness 500. Thus, as with the example of FIGS. 33 to 34, in the example of FIG. 35, the zone of weakness may break or open as the accessory channels 16, 18 move out of the interior cavities 56 of the circumferentially uncontinuous ribs 50. In some embodiments, each of the plurality of perforations or notches may have a length of 0.2 to 1.0 mm, spaced at an interval of 0.5 to 1.0 mm.

The sleeves 100, 200’, 200’’, 300, and 400 described above have been shown and described with reference to exemplary bendable endoscope systems, wherein bending of the distal portion 12 of each exemplary endoscope system is facilitated, at least in part, by a plurality of continuous ribs 40 and/or circumferentially uncontinuous ribs 50, with each of the circumferentially uncontinuous ribs 50 having a rib opening 51 to enable the working channel(s) 16, 18 to move in to and/or out of the interior cavities 56 of the circumferentially uncontinuous ribs 50. However, those skilled in the art will appreciate that the present disclosure is not limited to bendable endoscope systems having a plurality of continuous ribs 40 and/or circumferentially uncontinuous ribs 50. For example, the sleeves 100, 200’, 200’’, 300, and 400 may also be utilized in other endoscope systems wherein a bendable distal portion 12 has one or more structures that, alone or in combination, facilitate bending of the distal portion 12, wherein the one or more structures define an elongated opening, oriented in the longitudinal direction, to enable the working channel(s) 16, 18 to move in to and out of the bendable distal portion 12 through the elongated opening. The sleeves 100, 200’, 200’’, 300, and 400 described above can therefore also be applied to these other bendable distal portions 12, and operate in the same or similar manner as described above.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

What is claimed is:

1. A scope system, comprising:
an elongate tube comprising a lumen extending therethrough, the elongate tube further comprising a distal portion having a plurality of circumferentially uncontinuous ribs, each of the circumferentially uncontinuous ribs comprising a rib opening;
at least one accessory channel comprising a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration; and
a sleeve circumferentially surrounding at least the plurality of circumferentially discontinuous ribs, the sleeve having a longitudinal slot corresponding to the rib openings of the plurality of circumferentially discontinuous ribs,
wherein, during rotation of the distal end section relative to the distal portion, the at least one accessory channel is reversibly removable from an interior cavity of the plurality of circumferentially uncontinuous ribs through the rib openings and the longitudinal slot.

2. The scope system of claim 1, wherein a proximal end of the longitudinal slot comprises a generally semi-circular shape.

3. The scope system of claim 1, further comprising a patch fixed to and moveable with the at least one accessory channel, wherein when the at least one accessory channel is positioned in the interior cavity of the plurality of circumferentially uncontinuous ribs, the patch is positioned in at least a portion of the slot, and an external surface of the patch is flush with an external surface of the sleeve.

4. The scope system of claim 3, wherein a size and shape of the patch matches a size and shape of the slot.

5. The scope system of claim 1, further comprising a patch fixed to and moveable with the at least one accessory channel, wherein when the at least one accessory channel is positioned in the interior cavity of the plurality of circumferentially uncontinuous ribs, the patch is positioned in at least a portion of the slot, and at least a portion of the patch overlaps with a portion of the sleeve.

6. The scope system of claim 1, wherein a width of the slot is greater than or equal to an outer diameter of the at least one accessory channel.

7. The scope system of claim 1, wherein a width of the slot is greater than or equal to a width of the rib openings of the plurality of circumferentially uncontinuous ribs.

8. The scope system of claim 1, wherein a length of the longitudinal slot spans at least two of the circumferentially uncontinuous ribs.

9. The scope system of claim 1, wherein a length of the longitudinal slot spans at least 50% of a total number of the circumferentially uncontinuous ribs in the distal portion.

10. A scope system, comprising:
an elongate tube comprising a lumen extending therethrough, the elongate tube further comprising a distal portion having a plurality of circumferentially uncontinuous ribs, each of the circumferentially uncontinuous ribs comprising a rib opening;
at least one accessory channel comprising a tubular structure having an accessory lumen extending therethrough, the at least one accessory channel movably disposed at least partially within the lumen and the plurality of circumferentially uncontinuous ribs, the at least one accessory channel further comprising a distal end section rotatable relative to the distal portion of the elongate tube between a forward-facing configuration and a side-facing configuration; and
an inner cover surrounding at least a portion of a periphery of the at least one accessory channel positioned within the plurality of circumferentially uncontinuous ribs;
wherein, during rotation of the distal end section relative to the distal portion, the at least one accessory channel and the inner covering are reversibly removable from an interior cavity of the plurality of circumferentially uncontinuous ribs through the rib openings.

11. The scope system of claim 10, wherein the inner cover completely surrounds the periphery of the at least one accessory channel positioned within the plurality of circumferentially uncontinuous ribs.

12. The scope system of claim 10, wherein the inner cover partially surrounds the periphery of the at least one accessory channel positioned within the plurality of circumferentially uncontinuous ribs.

13. The scope system of claim 10, wherein a length of the inner cover spans at least two of the circumferentially uncontinuous ribs.

14. The scope system of claim 10, wherein a length of the inner cover spans at least 50% of the total number of the circumferentially uncontinuous ribs in the distal portion.

15. A scope system, comprising:
an elongate tube comprising a lumen extending therethrough, the elongate tube further comprising a bendable distal portion having an elongated opening oriented in a longitudinal direction;
at least one accessory channel comprising a tubular structure having an accessory lumen extending therethrough, the at least one accessory channel movably disposed at least partially within the lumen and the bendable distal portion, the at least one accessory channel further comprising a distal end section rotatable relative to the bendable distal portion of the elongate tube between a forward-facing configuration and a side-facing configuration; and
a sleeve circumferentially surrounding at least a portion of the bendable distal portion, the sleeve having a longitudinal slot corresponding to the elongated opening of the bendable distal portion,
wherein, during rotation of the distal end section relative to the bendable distal portion, the at least one accessory channel is reversibly removable from an interior cavity of the bendable distal portion through the elongated opening of the bendable distal portion and the longitudinal slot.

16. The scope system of claim 15, further comprising a patch fixed to and moveable with the at least one accessory channel, wherein when the at least one accessory channel is positioned in the interior cavity of the bendable distal portion, the patch is positioned in at least a portion of the slot, and an external surface of the patch is flush with an external surface of the sleeve.

17. The scope system of claim 16, wherein a size and shape of the patch matches a size and shape of the slot.

18. The scope system of claim 15, further comprising a patch fixed to and moveable with the at least one accessory channel, wherein when the at least one accessory channel is positioned in the interior cavity of the bendable distal portion, the patch is positioned in at least a portion of the slot, and at least a portion of the patch overlaps with a portion of the sleeve.

19. The scope system of claim 15, wherein a width of the slot is greater than or equal to an outer diameter of the at least one accessory channel.

20. The scope system of claim 15, wherein a width of the slot is greater than or equal to a width of the elongated opening of the bendable distal portion.
